(19) United States
(12) Patent Application Publication
(10) Pub. No.: US 2011/0110923 A1
(43) Pub. Date: May 12, 2011

(54) FISH ASSAY FOR EML4 AND ALK FUSION IN LUNG CANCER

(75) Inventors: Charles Lee, Marlborough, MA (US); Carly Murphy, Roxbury, MA (US); Pasi Janne, Newton, MA (US)

(73) Assignee: The Brigham and Women's Hospital, Inc., Boston, MA (US)

(21) Appl. No.: 12/867,336

(22) PCT Filed: Feb. 12, 2009
(86) PCT No.: PCT/US09/00879

§ 371 Date: Jan. 3, 2011

Related U.S. Application Data
(63) Continuation-in-part of application No. 61/065,422, filed on Feb. 12, 2008.

(51) Int. Cl. A61K 39/395; C12Q 1/68; A61K 38/00; A61P 35/00; A61K 31/7088; C07H 21/04

(52) U.S. Cl. 424/130.1; 435/6; 514/1.1; 514/44 R; 514/44 A; 536/24.3

(57) ABSTRACT

Methods and compositions provided relate to conducting a FISH assay for detecting a chromosomal inversion between EML4 and ALK. The FISH assay described herein is useful for diagnostic and prognostic purposes, as well as for determination of therapeutic strategies.

### RELATED APPLICATIONS

This application claims the benefit under 35 U.S.C. §119(e) of U.S. Provisional Application Ser. No. 61/065,422, entitled “FISH Assay for EML4 and ALK Fusion in Lung Cancer,” filed on Feb. 12, 2008, which is herein incorporated by reference in its entirety.

### GOVERNMENT INTEREST

This work was funded in part by the National Institutes of Health under grant number 1RO1CA114465-01. The government has rights in this invention.

### FIELD OF THE INVENTION

The invention pertains to a FISH assay to test for an inversion on chromosome 2 involving the EML4 and ALK genes. This assay has diagnostic and prognostic applications for lung cancer.

### BACKGROUND OF THE INVENTION

Anaplastic lymphoma kinase (ALK) was originally discovered from chromosomal translocations leading to the production of a fusion protein consisting of the C-terminal kinase domain of ALK and the N-terminal parts of various gene products (Morris et al., Science, 1994, 263:1281-1284). Translocations involving the ALK gene have been identified in 40-60% of anaplastic lymphomas (Falini et al., Am J Pathol, 1998, 153: 875-886) but they have also been discovered in some rare malignancies such as B-cell lymphomas, neuroblastomas, and myofibroblastic tumors (Gascoyne et al., Blood, 2003, 102: 2568-2573; Griffin et al., Cancer Res, 1999, 59: 2776-2780; Lawrence et al., Am J Pathol, 2000, 157: 377-384). Nucleophosmin (NPM) is the most common fusion partner of ALK (80% of translocations) but at least six other fusion partners have been identified (Amin et al., Blood, 2007, 110: 2259-2267). Expression of full length ALK is mainly restricted to developing neural tissues but some rare malignancies also express the gene (Falini et al., Am J Pathol, 1998, 153:875-886; Iwahara et al., Oncogene, 1997, 14: 439-449; Lamant et al., Am J Pathol, 2000, 156:1711-1721). Gene fusions involving ALK frequently lead to constitutive activation of ALK tyrosine kinase. Various downstream targets for ALK kinase activity have been identified including phosphatidylinositol 3-kinase (PI3K), STAT3, phospholipase γ, and extracellular regulated kinase 1/2 (ERK1/2) (Amin et al., Blood, 2007, 110: 2259-2267).

The fusion of the ALK gene with echinoderm microtubule-associated protein-like 4 (EML4) has recently been detected in a subset of Japanese non-small cell lung cancers (NSCLC) (Soda et al., Nature, 2007, 448: 561-566). The frequency of EML4-ALK fusions was found to be 6.7% (5/75) in Japanese NSCLCs. The EML4-ALK fusions were detected in adenocarcinoma and squamous cell carcinomas, from never or current smokers, in both genders, and were mutually exclusive with EGFR or K-Ras mutations. Furthermore, EML4-ALK was transforming in 3T3 cells and in Ba/F3 models (Soda et al., Nature, 2007, 448:561-566).

### SUMMARY OF INVENTION

Described herein are methods and compositions for performing a FISH assay for the detection of a chromosomal inversion involving EML4 and ALK. Also included are methods for diagnosing and prognosing non-small cell lung cancer (NSCLC) based at least in part on a fluorescent in situ hybridization (FISH) assay to detect an EML4-ALK chromosomal inversion, and methods for treating diseases characterized by expression of an EML4-ALK inversion using compositions that inhibit ALK kinase activity. Further aspects of the invention relate to methods for determining whether subjects with NSCLC should be treated with a composition that inhibits ALK kinase activity, based at least in part on analysis of whether such subjects exhibit an EML4-ALK chromosomal inversion. Probes for use in a FISH assay to detect an EML4-ALK chromosomal inversion, and kits for performing such a FISH assay are also described.

Aspects of the invention relate to methods for performing a FISH assay to identify an EML4-ALK inversion within a chromosomal preparation, involving: contacting, under hybridization conditions, a chromosomal preparation with a set of probes comprising a first nucleic acid probe and a second nucleic acid probe; wherein the first probe has a first label and is hybridizable to an uninverted form of the first chromosome; wherein the second probe has a second label different from the first label and is hybridizable to an uninverted form of the second chromosome; wherein (i) if the first and second chromosomes have undergone an inversion and fusion, the first and second probes hybridize to a derivative chromosome formed via the inversion and fusion, such that both the first and second labels appear as a single signal; whereas (ii) if the first and second chromosomes have not undergone the inversion and fusion, the first and second probes hybridize to their respective chromosomes, such that two signals are detected; detecting a pattern of hybridization for the first and second probes; and determining from the pattern whether the first and second probes appear on the derivative chromosome or appear separately on the first and second chromosomes, respectively, thereby determining the presence or absence of the EML4-ALK inversion. In some embodiments each nucleic acid probe is hybridizable to an uninverted form of each chromosome at a region located within 5 Mb, 2 MB or 1 Mb of the breakpoint associated with the inversion.

In some embodiments, methods for determining the presence or absence of an EML4-ALK inversion involve: contacting, under hybridization conditions, a chromosomal preparation with a set of probes comprising a first nucleic acid probe comprising at least 80% sequence identity with the sequence of RP11-667I6 and having a first label and being hybridizable to a first chromosome, and a second nucleic acid probe comprising at least 80% sequence identity with the sequence of RP11-100C1 and having a second label different from the first label and being hybridizable to a second chromosome; wherein (i) if the first and second chromosomes have undergone an inversion and fusion, the first and second probes hybridize to a derivative chromosome formed via the inversion and fusion, such that both the first and second labels appear as a single signal; whereas (ii) if the first and second chromosomes have not undergone the inversion and fusion, the first and second probes hybridize to their respective chromosomes, such that two signals are detected; detecting a pattern of hybridization for the first and second probes; determining from the pattern whether the first and second probes appear on the derivative chromosome or appear separately on the first and second chromosomes, respectively, thereby determining the presence or absence of the EML4-ALK inversion. In some embodiments the first nucleic acid probe is RP11-667I6, and has a label, and the second nucleic acid probe is RP11-100C1, and has a second label.

Further described herein are methods for diagnosing NSCLC in a subject, comprising: isolating a biological sample from the subject; generating a chromosomal preparation from the sample; performing FISH on the chromosomal preparation to identify the presence or absence of an EML4-ALK inversion; and determining that the subject has NSCLC if the chromosomal preparation contains an EML4-ALK inversion. In some embodiments each nucleic acid probe is hybridizable to an uninverted form of each chromosome at a region located within 5 Mb, 2 MB or 1 Mb of the breakpoint associated with the inversion. In certain embodiments the first nucleic acid probe has at least 80% sequence identity with the sequence of RP11-667I6 and has a first label, and the second nucleic acid probe has at least 80% sequence identity with the sequence of RP11-100C1, and has a second label. Aspects of the invention include methods of classifying the subject as exhibiting a poor, intermediate or good prognosis based on the results of the FISH analysis. In some embodiments of the diagnosis and/or prognosis of NSCLC, the NSCLC is adenocarcinoma. In other embodiments of the diagnosis and/or prognosis of NSCLC, the NSCLC is squamous cell carcinoma.

Aspects of the invention further relate to methods for determining whether a subject with NSCLC should be treated with a composition that inhibits ALK kinase activity, the method involves: isolating a biological sample from the subject; generating a chromosomal preparation from the sample; performing FISH on the chromosomal preparation to identify the presence or absence of an EML4-ALK inversion; and determining that the subject should be treated with a composition that inhibits ALK kinase activity if the chromosomal preparation contains an EML4-ALK inversion. In some embodiments, the method further involves treating the subject with a composition that inhibits ALK kinase activity. In some embodiments, an EGFR inhibitor is also administered to the subject. In some embodiments each nucleic acid probe is hybridizable to an uninverted form of each chromosome at a region located within 5 Mb, 2 MB or 1 Mb of the breakpoint associated with the inversion. In some embodiments the first nucleic acid probe has at least 80% sequence identity with the sequence of RP11-667I6 and has a first label, and the second nucleic acid probe has at least 80% sequence identity with the sequence of RP11-100C1, and has a second label. In some embodiments the composition that inhibits ALK kinase activity is a kinase inhibitor such as NVP-TAE684 or PF-02341066. In some embodiments the composition that inhibits ALK kinase activity comprises an agent that knocks down expression of ALK. In certain embodiments the composition that inhibits ALK kinase activity is an antisense RNA, an RNAi, a ribozyme, an antibody, a small molecule, a peptide, an aptamer or any combination thereof.

Aspects of the invention further include nucleic acid probes for detecting a chromosomal inversion between EML4 and ALK. In some embodiments the nucleic acid probe has a label and hybridizes to an EML4 chromosome, such that if EML4 has not undergone an inversion the probe will hybridize to the uninverted form of the EML4 chromosome, and if EML4 has undergone an inversion the probe will hybridize to the derivative chromosome formed via the inversion. In some embodiments the nucleic acid probe has a label and hybridizes to an ALK chromosome, such that if ALK has not undergone an inversion the probe will hybridize to the uninverted form of the ALK chromosome, and if ALK has undergone an inversion the probe will hybridize to the derivative chromosome formed via the inversion. In some embodiments each nucleic acid probe is hybridizable to an uninverted form of each chromosome at a region located within 5 Mb, 2 MB or 1 Mb of the breakpoint associated with the inversion. In some embodiments the probe comprises at least 80% sequence identity with the sequence of RP11-667I6 and has a label. In some embodiments the probe comprises at least 80% sequence identity with the sequence of RP11-100C1 and has a label.

Further described herein are kits for identifying an EML4-ALK inversion within a chromosomal preparation, the kit includes: a first nucleic acid probe wherein the probe has a label and hybridizes to an EML4 chromosome, such that if EML4 has not undergone an inversion the probe will hybridize to the uninverted form of the EML4 chromosome, and if EML4 has undergone an inversion the probe will hybridize to the derivative chromosome formed via the inversion; a second nucleic acid probe wherein the probe has a label and hybridizes to an ALK chromosome, such that if ALK has not undergone an inversion the probe will hybridize to the uninverted form of the ALK chromosome, and if ALK has undergone an inversion the probe will hybridize to the derivative chromosome formed via the inversion; instructions for use of the first and second probes for performing a FISH assay to identify an EML4-ALK inversion within a chromosomal preparation.

In some embodiments each nucleic acid probe is hybridizable to an uninverted form of each chromosome at a region located within 5 Mb, 2 MB or 1 Mb of the breakpoint associated with the inversion. In some embodiments the kit further comprises a DNA counterstain, such as DAPI. In some embodiments the kit further comprises components such as hybridization buffer, mounting media, and a control slide.

Further aspects of the invention involve methods for treating a disease characterized by expression of an EML4-ALK inversion in a subject that has been diagnosed as having an EML4-ALK inversion, by administering to the subject a composition that inhibits ALK kinase activity. In some embodiments the disease is cancer, such as non-small cell lung cancer. In certain embodiments the non-small cell lung cancer is adenocarcinoma or squamous cell carcinoma.

In some embodiments the composition that inhibits ALK kinase activity includes a kinase inhibitor such as NVP-TAE684 or PF-02341066. In some embodiments the composition that inhibits ALK kinase activity includes an agent that knocks down expression of ALK such as an antisense RNA, an RNAi, a ribozyme, or any combination thereof. In some embodiments the composition that inhibits ALK kinase activity includes an antibody, a small molecule, a peptide, an aptamer or any combination thereof. Methods described herein for treating a disease characterized by expression of an EML4-ALK inversion in a subject that has been diagnosed as having an EML4-ALK inversion can also include administering an EGFR inhibitor such as Erlotinib, Gefitinib, or AG 1478. Methods described herein may also include administering a chemotherapeutic agent. In some embodiments a plurality of ALK kinase inhibitors and/or EGFR inhibitors and/or chemotherapeutic agents are administered. In some embodiments the methods of treating a disease characterized by expression of an EML4-ALK inversion in a subject that has been diagnosed as having an EML4-ALK inversion, are applied to a subject who has been diagnosed as having an EML4-ALK inversion using diagnostic methods described herein. In some embodiments the subject is diagnosed as having an EGFR mutation.

The subject may in some embodiments undergo surgery and/or radiation therapy.

Aspects of the invention relate to inhibiting ALK kinase activity in a cell which expresses an EML4-ALK inversion, by contacting the cell with a composition that inhibits ALK kinase activity.

### BRIEF DESCRIPTION OF DRAWINGS

FIG. 1 demonstrates EML4-ALK in NSCLC cell lines and tumors. FIG. 1A shows the detection of ALK fusion genes in lung cancer cell lines using exon arrays. In the screen of 83 lung cancer cell lines (80/83 NSCLCs), exon arrays showed that H3122 and H2228 cell lines had significantly higher signal (log 2 difference) for ALK probes #80-140 corresponding to exons 20-29 of ALK compared with other 81 cell lines. Probes were assigned into three categories based on their labeling intensity; non-responsive probes (light shade), low-intensity probes (intermediate shade), high-intensity probes (dark shade). Only high-intensity probes were used in breakpoint detection. FIG. 1B shows RT-PCR detection of EML4-ALK fusion in NSCLC cell lines and tumors. Primer set 2 amplifies EML4-ALK fusion genes from H3122, H2228, and DFCI032 cell lines but not from A549 line. Primer sets 1 and 2 also detected EML4-ALK fusion from 8 primary NSCLCs. H3122 cell serves as positive control, A549 as negative control. FIG. 1C shows a schematic representation of the four different EML4-ALK variants in NSCLC.

FIG. 2 shows detection of EML4-ALK using FISH. FIG. 2A shows a wild type PC-9 cell line, wherein signals for ALK (arrow) and EML4 (arrowhead) are seen separately. FIG. 2B shows an H2228 cell line, wherein the fusion signal of EML4-ALK (arrow) is seen in a small extra-chromosomal fragment (arrow). FIG. 2C shows a DFCI032 cell line, wherein one of the chromosomes shows an EML4-ALK fusion signal in yellow (arrow). FIG. 2D shows Interphase FISH for EML4-ALK from an FFPE specimen obtained from the tumor of the patient whose pleural effusion was used to establish the DFCI032 cell line shown in FIG. 2C. The tumor is heterozygous for the EML4-ALK fusion signal (arrow).

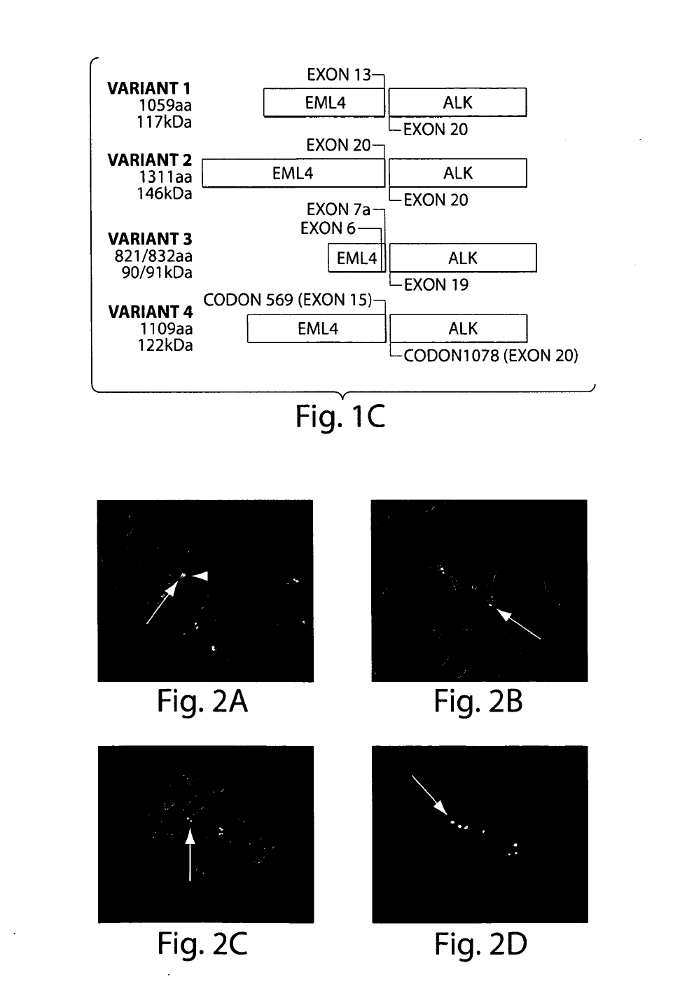

FIG. 3 shows the effect of NVP-TAE684 on growth of EML4-ALK containing NSCLC cell lines. FIG. 3A shows a graph indicating NSCLC cells that were treated with TAE-684 at the indicated concentrations. Viable cells were measured after 72 hours of treatment. The percentage of viable cells is shown relative to untreated controls. A549 (KRAS G12S); PC9 (EGFR delE746_A750); H2228 (EML4-ALK variant 3); H3122 (EML4-ALK variant 1); DFCI032 (EML4-ALK variant 1). FIG. 3B shows FACS analysis of sub G1 fraction without treatment (left bar) and after treatment with 0.1 μM NVP-TAE684 for 72 h (right bar). Significant apoptosis following TAE-684 treatment is only observed in the H3122 cell line. FIG. 3C shows Western blot analysis of PARP following treatment with 0.1 μM NVP-TAE684 for 72 h. The 89 kDa cleaved PARP products is observed only in the H3122 cell line consistent with the effects of TAE-684 on cell growth in A.

FIG. 4 shows Western blot analysis following NVP-TAE684 treatment in wild type and EML4-ALK positive NSCLC cell lines. Total and phosphorylated ALK are only detected in EML4-ALK positive cell lines (H3122, H2228, DFCI032) but not in wild type control (PC-9). In H3122 and DFCI032 cell lines, ALK positive band migrates at ˜115 kDa corresponding to predicted molecular weight (117 kDa) of the variant 1 (arrow 1) while in H2228, the band migrates at ˜90 kDa which also corresponds to the predicted molecular weight (90/91 kDa) of the variant 3 (arrow 3). ALK phosphorylation is completely inhibited following 0.1 μM NVP-TAE684 treatment (6 hours) in all the cell lines. Phosphorylation of Akt, STAT3, and ERK1/2 decrease in H3122 and H2228 cell lines with NVP-TAE684 but remain unchanged in DFCI032 and PC-9 lines. All the cell lines show presence of PTEN. α-tubulin is used as a loading control.

FIG. 5 shows expression levels of different parts of ALK gene in NSCLC cell lines using exon arrays. Non-responsive probes are indicated by a light shade, low-intensity probes are indicated by an intermediate shade, and high-intensity probes are indicated by a dark shade. FIG. 5A shows that in H2228 cell line, exon arrays detected significantly higher signal for the ALK probes located at the 3′ end compared to rest of the 83 cell lines. FIG. 5B shows that in H3122 cell line, analogously to H2228 line, significantly higher signal was seen in the 3′ end of the ALK. FIG. 5C shows that no differences in the signals for the different ALK probes in the HCC2935 cell line.

FIG. 6 shows an illustration of the FISH method used in detecting EML4-ALK. In wild type specimens, signals for ALK and EML4 are seen separately in chromosome 2. When EML4 and ALK are fused through inversion (inv(2)(p23.3p21), both individual signals fuse and form a single signal.

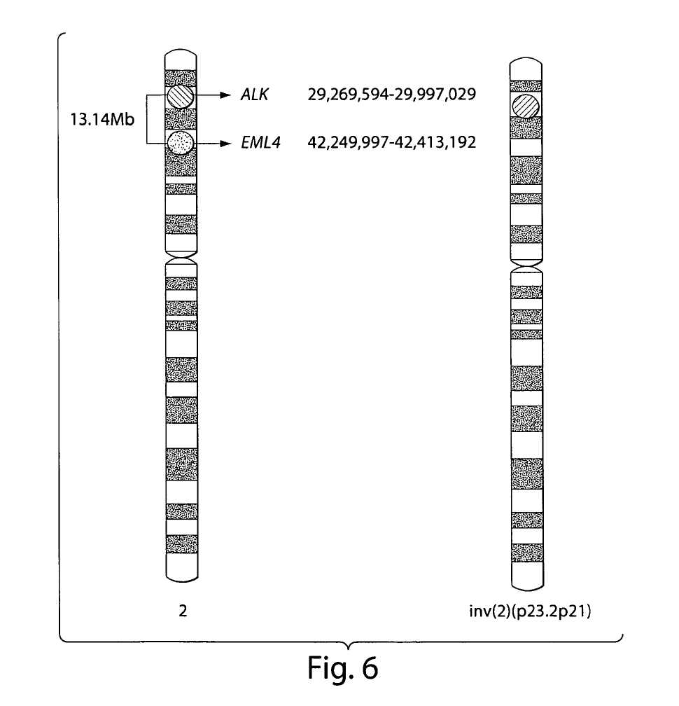

FIG. 7 depicts a kit comprising nucleic acid probes and instructions for their use in a FISH assay.

### DETAILED DESCRIPTION

Aspects of the invention relate to methods and compositions for detecting an abnormal chromosomal inversion bringing together EML4 and ALK. The invention relates, at least in part, to the discovery that a fluorescent in situ hybridization assay (FISH) can be used to detect a chromosomal inversion that results in an EML4-ALK inversion. The prevalence of this chromosomal inversion in non-small cell lung cancer (NSCLC) leads to diagnostic and prognostic applications for the FISH assay described herein. Use of the FISH assay for detection of an EML4-ALK inversion also has applications for determining appropriate treatment strategies for subjects who exhibit such a gene fusion. Further described herein are probes for use in a FISH assay for detecting an EML4-ALK inversion, methods for generating such probes, and kits containing such probes.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the invention relate to the use of fluorescent in situ hybridization. As used herein “fluorescent in situ hybridization” or “FISH” refers to a method for detecting or localizing a specific DNA sequence on a chromosome through the use of a labeled nucleic acid probe that hybridizes to a specific DNA sequence on a chromosome. As used herein a “nucleic acid probe” refers to a nucleic acid (such as DNA, RNA, PNA etc.) sequence that recognizes and hybridizes to a specific DNA sequence on a chromosome. While FISH has many diverse research applications, one application pertains to the ability to detect chromosomal inversions. As used herein a “chromosomal inversion” refers to a rearrangement in which a segment of a chromosome is reversed end to end. An inversion can result in a fusion between two genes that does not normally occur in a wild-type or normal cell, and can lead to a disorder such as cancer. Thus, detection of a chromosomal inversion in a cell can be indicative of a disorder such as cancer.

Chromosomal inversions involving the Anaplastic lymphoma kinase (ALK) gene are detected in multiple types of cancer, and can involve fusions with at least six different genes (Amin et al., Blood, 2007, 110:2259-2267). In these inversions, the C-terminal kinase domain of ALK is fused to the N-terminal regions of various genes (Morris et al., Science, 1994, 263:1281-1284). Recently a chromosomal inversion was detected in Japanese NSCLC patients, in which the ALK gene is fused to the echinoderm microtubule-associated protein-like 4 (EML4) gene (Soda et al., Nature, 2007, 448: 561-566). The chromosomal inversion or fusion between EML4 and ALK is referred to herein as EML4-ALK. Data presented herein in the Example section reveals that the EML4-ALK fusion is also prevalent in American and Korean NSCLC patients.

Aspects of the invention relate to the use of FISH in detecting the presence of an EML4-ALK chromosomal inversion. Methods described herein comprise contacting, under hybridization conditions, a chromosomal preparation with a set of probes comprising a first nucleic acid probe and a second nucleic acid probe; wherein the first probe has a first label and is hybridizable to an uninverted form of the first chromosome; wherein the second probe has a second label different from the first label and is hybridizable to an uninverted form of the second chromosome; wherein (i) if the first and second chromosomes have undergone an inversion and fusion, the first and second probes hybridize to a derivative chromosome formed via the inversion and fusion, such that both the first and second labels appear as a single signal; whereas (ii) if the first and second chromosomes have not undergone the inversion and fusion, the first and second probes hybridize to their respective chromosomes, such that two signals are detected; detecting a pattern of hybridization for the first and second probes; and determining from the pattern whether the first and second probes appear on the derivative chromosome or appear separately on the first and second chromosomes, respectively, thereby determining the presence or absence of the EML4/ALK inversion.

It should be appreciated that multiple different approaches can be used to design probe sets compatible with the instant invention. In some embodiments the nucleic acid probes may be described as “fusion probes.” In these embodiments, a first probe has a label and hybridizes to a chromosome comprising EML4, such that if EML4 has not undergone an inversion the probe will hybridize to the uninverted form of the chromosome comprising EML4, and if EML4 has undergone an inversion the probe will hybridize to the derivative chromosome formed via the inversion. The second nucleic acid probe has a different label from the first nucleic acid probe and hybridizes to a chromosome comprising ALK, such that if ALK has not undergone an inversion the probe will hybridize to the uninverted form of the chromosome comprising ALK, and if ALK has undergone an inversion the probe will hybridize to the derivative chromosome formed via the inversion. In a wild-type chromosomal preparation, wherein EML4 and ALK have not fused due to an inversion, the first and second probes hybridize to their respective uninverted chromosomal regions, and two separate signals are detected. In an abnormal chromosomal preparation wherein EML4 and ALK have fused due to an inversion, the first and second probes hybridize to a derivative chromosome formed via the inversion and fusion, such that both the first and second labels appear as a single signal.

In other embodiments, “break-apart” probes are also be compatible with the instant invention. In these embodiments the first probe has a label and is hybridizable to an uninverted form of a chromosome near an inversion breakpoint. The second probe has a different label from the first probe and is hybridizable to a region of the same chromosome on the other side of the inversion breakpoint. In a normal wild-type chromosomal preparation, wherein the chromosome has not undergone an abnormal inversion, the two probes will be detected as adjacent signals, or a single signal. In an abnormal chromosomal preparation wherein the chromosome has undergone an inversion and fusion, the two signals will be detected apart, as distinct signals, due to their separation via the chromosomal inversion. Break-apart probes for either EML4 or ALK are compatible with the instant invention.

In some embodiments a nucleic acid probe for use in a FISH assay, is generated from a BAC (bacterial artificial chromosome) clone, such as one available from the BAC PAC Resources Center (BPRC) at the Children's Hospital Oakland Research Institute, Oakland, Calif. As used herein a “BAC” refers to a vector used to clone DNA fragments in Escherichia coli cells. BACs typically contain 50-300 kb DNA inserts. In other embodiments a nucleic acid probe for use in a FISH assay can be generated from a fosmid. As used herein a “fosmid” refers to a cloning vector based on the bacterial F-plasmid. In certain embodiments a probe is generated from multiple fosmids that are pooled together. It should be appreciated that the length of an optimal probe for a FISH assay may need to be empirically determined. In some embodiments the length of a probe is between 80-150 kbp. It should further be appreciated that other sources of large DNA fragments would also be compatible with probe generation for FISH assays, and accordingly, with methods of the instant invention. Smaller DNA fragments are also compatible with methods of the instant invention, and in some embodiments are pooled together (as described above for fosmids).

According to aspects of the invention, a nucleic acid probe is labeled with a tag or label. In some embodiments the tag or label for use in a FISH assay is a fluorescent tag or label, also referred to as a fluorophor. Any appropriate technique for labeling a nucleic acid, as would be understood by one of ordinary skill in the art, is compatible with the instant invention. In some embodiments the nucleic acid probe is labeled through nick translation, according to standard protocols. In other embodiments the nucleic acid probe is labeled through random priming, according to standard protocols. In further embodiments the nucleic acid probe is labeled through end labeling, according to standard protocols. It should be appreciated that any tag or label that can be used to label a nucleic acid probe may be compatible with the instant invention. In some embodiments the tag is selected from, but is not limited to, SpectrumRed-dUTP, SpectrumGreen-dUTP, SpectrumGreen-11-dUTP, and SpectrumOrange-dUTP, all available from Abbott Molecular, Des Plaines, Ill. In some embodiments a probe may be labeled with biotin or digoxigenin.

In some embodiments the nucleic acid probe is hybridizable to an uninverted form of the chromosome at a region located within 5 Mb, 2 Mb, 1 Mb, or less than 1 Mb of the breakpoint associated with the inversion. ALK and EML4 are both located in the short arm of chromosome 2, separated by 12 megabases, and are in opposite 5′ to 3′ orientations. Two different variants of EML4-ALK fusions have previously been characterized, both involving exons 20-29 of ALK fused to exon 1-13 (variant 1) or 1-20 (variant 2) of the EML4 gene (Soda et al., Nature, 2007, 448: 561-566). Variant 3 (wherein EML4 exon 6 is fused to ALK exon 20), alters the splicing of the EML4 part of the fusion to incorporate an alternatively spliced 33 bp fragment (exon 7a). Variant 4 of the fusion gene, described herein, fuses EML4 codons 1-569 (exon 15) to codons 1078-1621 of ALK. In variant 1 of the EML4-ALK fusion, the breakpoint is 3.6 kb downstream of exon 13 of EML4, and 297 by upstream of exon 21 of ALK. In variant 2 of the EML4-ALK fusion, the breakpoint is located 545 by downstream of exon 20 of EML4 and 232 by upstream of exon 21 of ALK. In variants 3 and 4 of the EML4-ALK fusion, the exact breakpoints have not yet been determined. However, the breakpoints of specific variants can be determined by those of ordinary skill in the art.

It should be appreciated that multiple probes are compatible with the instant invention. Probes can be designed based on the information provided herein regarding inversion breakpoints and sequence data for EML4-ALK fusion variants, as well as publicly available human genome sequence data, such as that available through the UCSC genome database website. It should also be appreciated that further variants of EML4-ALK fusions may exist. Probes and assays described herein encompass all possible variants of EML4-ALK, and methods for identifying these variants. In some embodiments probes that are designed to recognize an EML4-ALK inversion will recognize all variants of the EML4-ALK inversions. In other embodiments probes that are designed to recognize an EML4-ALK inversion may recognize one or several variants of the EML4-ALK inversions.

In some embodiments a nucleic acid probe that hybridizes to the 3′ end of ALK, and a nucleic acid probe that hybridizes to the 5′ end of EML4 are used for FISH to detect an EML4-ALK inversion. In some embodiments, the nucleic acid probe that hybridizes to the 3′ end of ALK is generated from the BAC clone RP11-100C1, and has a label. In some embodiments the nucleic acid probe that hybridizes to the 3′ end of ALK comprises at least 80%, 85%, 90%, 95%, 96%, 97%, 98% or 99% sequence identity with RP11-100C1. In some embodiments a nucleic acid probe that hybridizes to the 5′ end of EML4 is generated from the BAC clone RP11-667I6, and has a label. In some embodiments the nucleic acid probe that hybridizes to the 5′ end of EML4 comprises at least 80%, 85%, 90%, 95%, 96%, 97%, 98% or 99% sequence identity with RP11-667I6. Both RP11-100C1 and RP11-667I6 are available from the BAC PAC Resources Center (BPRC) at the Children's Hospital Oakland Research Institute, Oakland, Calif.

According to aspects of the invention, FISH is performed on a chromosomal preparation using fluorescently labeled probes such as a first probe comprising at least 80% sequence identity with RP11-100C1 and a second probe comprising at least 80% sequence identity with RP11-667I6. FISH is performed according to standard techniques familiar to one of ordinary skill in the art (Lee et al., Chromosoma, 2000, 109:381-389). In some embodiments, sample preparation for performing FISH involves reduction of autofluorescence and mounting of a sample on a slide. A sample may in some embodiments consist of a fixed and treated tissue, a frozen sample, a sample on a slide, or a sample that is paraffin embedded. Aspects of sample preparation are discussed further in US Patent Publications US2006/0199213 and US2005/0100944, incorporated herein by reference.

FISH assays rely on detecting hybridization between a nucleic acid probe and a specific DNA sequence on a chromosome. As used herein “hybridization” refers to the process of joining two complementary strands of DNA or RNA, or hybrids thereof, to form a double-stranded molecule. The hybridization step may involve one, two, or multiple probes. It will be appreciated that hybridization conditions may need to be determined empirically for different probes. Hybridization conditions can be varied, producing a range of high to low stringency conditions, as will be known to those of ordinary skill in the art. Several factors can be manipulated experimentally in order to optimize hybridization, including but not limited to temperature, salt concentration, formamide concentration, and presence of other components such as dextran sulfate (as discussed in US Patent Publication US2005/0100944). These factors and others can be varied during hybridization steps and during subsequent wash steps in order to optimize hybridization signals for a given probe and sample. General conditions for in situ hybridization are discussed in Leitch et al., In Situ Hybridization: a practical guide, Oxford BIOS Scientific Publishers, Microscopy handbooks v. 27 (1994). Higher stringency conditions generally result in lower background signals for probe detection, but can also decrease sensitivity. In some embodiments high stringency conditions may consist of 0.1×SSPE, 0.1% SDS, 65° C.; medium stringency conditions may consist of 0.2×SSPE, 0.1% SDS, 50° C.; and low stringency conditions may consist of 1×SSPE, 0.1% SDS, 50° C. (as discussed in US Patent Publication US2006/0199213). It will be appreciated that many possible variations of these conditions, and many other components including a variety of buffers and salts will be compatible with the instant invention.

A positive hybridization signal in a FISH assay is detected by visualization of the tag accompanying the nucleic acid probe, through fluorescence microscopy. In some embodiments the first nucleic acid probe is tagged with a fluorescent tag such as SpectrumRed-dUTP, while the second nucleic acid probe is tagged with a different fluorescent tag such as SpectrumGreen-dUTP. In a chromosomal preparation where there has not been a chromosomal inversion between EML4 and ALK, the two fluorescent signals will be detected separately, as red and green signals. In a chromosomal preparation where there has been a chromosomal inversion between EML4 and ALK, the two fluorescent signals will merge and be detected as a single yellow signal. Thus the gene fusion will be indicated both by the proximity of the two signals following the inversion, and the detection of a different color due to the merging of the two fluorescent signals.

In some embodiments, a FISH assay will involve a test sample and a control sample. In some embodiments a control sample may be a wild-type or normal chromosomal preparation, while the test sample may be a sample in which the presence of a chromosomal inversion between EML4 and ALK is suspected. The test sample and control sample will be treated with the same probes, and the localization and fluorescent signals of the probes will be compared between the control and test samples. In embodiments where the control sample is a wild-type or normal chromosomal preparation, a similar localization and fluorescent signal between probes in a test sample and a control sample may indicate that the test sample does not contain an EML4-ALK inversion, while a difference in the localization and fluorescent signal between probes in a test sample and a control sample may indicate that the test sample does contain an EML4-ALK inversion. In other embodiments a control sample may be a sample which is known to contain an EML4-ALK inversion. In these embodiments a similar localization and fluorescent signal between probes in a test sample and a control sample may indicate that the test sample does contain an EML4-ALK inversion, while a difference in the localization and fluorescent signal between probes in a test sample and a control sample may indicate that the test sample does not contain an EML4-ALK inversion.

Aspects of the invention include methods for diagnosing or monitoring the onset, progression, or regression of cancer in a subject by, for example, obtaining cell or tissue samples from a subject and assaying such samples for the presence of the EML4-ALK inversion. As used herein, the term “cancer” refers to an uncontrolled growth of cells that may interfere with the normal functioning of the bodily organs and systems, and includes both primary and metastatic tumors. Primary tumors or cancers that migrate from their original location and seed vital organs can eventually lead to the death of the subject through the functional deterioration of the affected organs. A metastasis is a cancer cell or group of cancer cells, distinct from the primary tumor location, resulting from the dissemination of cancer cells from the primary tumor to other parts of the body. Metastases may eventually result in death of a subject.

In some embodiments a subject who is diagnosed or treated by aspects of the claimed invention, is a subject with lung cancer. Lung cancer encompasses both small cell lung cancer (SCLC) and non-small cell lung cancer (NSCLC). Non-small cell lung cancer, which is responsible for about 80% of cases of lung cancer, encompasses multiple types of cancer including but not limited to Squamous cell carcinoma (also called epidermoid carcinoma), Large cell carcinoma, Adenocarcinoma, Pleomorphic, Carcinoid tumor, Salivary gland carcinoma, and unclassified carcinoma. Aspects of the invention encompass all categories of NSCLC. In some embodiments the NSCLC is adenocarcinoma. In other embodiments the NSCLC is squamous cell carcinoma.

A subject may or may not be suspected of having cancer. Onset of a condition is the initiation of the physiological changes or characteristics associated with the condition in a subject. Such changes may be evidenced by physiological symptoms, or may be clinically asymptomatic. For example, the onset of cancer may be followed by a period during which there may be cancer-associated physiological characteristics in the subject, even though clinical symptoms may not be evident at that time. The progression of a condition follows onset and is the advancement of the physiological characteristics of the condition, which may or may not be marked by an increase in clinical symptoms. In contrast, the regression of a condition is a decrease in physiological characteristics of the condition, perhaps with a parallel reduction in symptoms, and may result from a treatment or may be a natural reversal in the condition.

The presence of a cancer-associated gene fusion such as the EML4-ALK fusion, that is not present in non-cancer cells or tissues, is determined to be a marker for cancer in the subject. The onset of a cancer condition may be indicated by the appearance of such a marker(s) in a subject's samples where there was no such marker(s) determined previously. For example, if marker(s) for cancer are determined not to be present in a first sample from a subject, the determination that cancer marker(s) are present in a second or subsequent sample from the subject is an indication of the onset of cancer in the subject. Some examples of cancer associated markers may be differentially expressed in primary tumors versus metastases, thereby allowing the stage and/or diagnostic level of the disease to be established, based on the identification of selected cancer-associated polypeptides in a subject sample.

Different types of cancer in a single tissue type may express different cancer-associated markers. Such variations may allow cancer-specific diagnosis and subsequent treatment tailored to the patient's specific condition. These differences in expression, can enable a physician to diagnose the cancer on the basis of differential expression of the cancer-associated markers, and permits specific treatments to be selected and administered on the basis of the differential markers. The isolation and identification of a cancer-associated marker such as the EML4-ALK inversion, permits the artisan to diagnose a disorder characterized by expression of this cancer-associated marker.

Aspects of the invention relate to diagnosis of NSCLC, through the use of a FISH assay for detection of an EML4-ALK inversion. In some embodiments a method for diagnosing NSCLC comprises isolating a biological sample from a subject, generating a chromosomal preparation from the sample, performing a FISH assay on the chromosomal preparation to identify the presence or absence of an EML4-ALK inversion, and determining that the subject has NSCLC if the chromosomal preparation contains an EML4-ALK inversion. A subject that has been diagnosed with NSCLC, through the use of a FISH assay for detection of an EML4-ALK inversion may be treated by administering a composition that inhibits ALK kinase activity. In some embodiments a composition comprising an EGFR inhibitor or other therapeutics may also be administered.

As used herein, the term “biological sample” may refer to a whole organism or a subset of its tissues, cells or component parts. A “biological sample” may also refer to a homogenate, lysate, or extract prepared from a whole organism or a subset of its tissues, cells or component parts, or a fraction or portion thereof. In some embodiments, a biological sample will be a sample from lung tissue. In some embodiments a biological sample may be in vivo. In other embodiments a biological sample may be in vitro. In some embodiments a biological sample may be a cell line, cell culture or cell suspension. Preferably, a biological sample corresponds to the amount and type of DNA and/or expression products present in a parent cell from which the sample was derived. A biological sample can be from a human or non-human subject. Chromosomal preparations are prepared from biological samples according to standard protocols. In some embodiments the sample used for performing FISH is a formalin fixed paraffin embedded (FFPE) specimen.

In some embodiments the results of the FISH analysis to detect the presence or absence of an EML4-ALK inversion, will be used in diagnosis of NSCLC. In other embodiments, the results of the FISH analysis to identify the presence or absence of an EML4-ALK inversion will be used in classification of the subject as exhibiting a poor, intermediate or good NSCLC prognosis based on the results of the FISH analysis. It should be appreciated that performance of a FISH assay to detect an EML4-ALK inversion for diagnosis or prognosis of NSCLC may be combined with analysis of other markers, or other diagnostic or prognostic assays. In some embodiments, other assays may be conducted in combination with, or following a FISH assay, for further confirmation, or for further analysis of the molecular basis of the chromosomal inversion. For example PCR or RT-PCR may be conducted on a biological sample to verify or confirm which variant of the EML4-ALK fusion is present in a biological sample. In some embodiments performance of an assay to measure ALK kinase activity may also be conducted on a biological sample that is suspected of containing, or known to contain an EML4-ALK inversion.

In some embodiments, a test sample may be a sample from a subject who has NSCLC or a precancerous condition, while a control sample may be a sample from a cell or subject that is free of cancer and/or free of a precancerous condition. In these embodiments, detection of an EML4-ALK inversion in the test sample but not in the control sample may indicate that the test sample came from a subject who has NSCLC or a precancerous condition. In some embodiments, a control sample may be a sample that is from a cell or subject that is known to have NSCLC or a precancerous condition exhibiting an EML4-ALK inversion. In these embodiments detection of an EML4-ALK inversion in the test sample and in the control sample may indicate that the test sample came from a subject who has NSCLC or a precancerous condition. In some embodiments a control sample may be an NSCLC cell line that does or does not contain an EML4-ALK inversion.

According to some aspects of the invention, a subject (e.g., an NSCLC patient) may be identified as a candidate for treatment with a composition that inhibits ALK kinase activity if the subject has a disease (e.g., NSCLC) that expresses an EML4-ALK fusion in at least some, if not all, of the cancer cells. Accordingly, in some embodiments a subject (e.g., an NSCLC patient) is tested for the presence of an EML4-ALK fusion, and if present, is identified as a candidate for treatment with a composition that inhibits ALK kinase activity. It should be appreciated that detection of any fusion between EML4 and ALK, encompassing any possible variant of this gene fusion, may be considered an indicator for treatment with an inhibitor of ALK kinase activity. In some embodiments, a subject (e.g., an NSCLC patient) who has a disease (e.g., NSCLC) that exhibits an EML4-ALK fusion in at least some, if not all, of the cancer cells, may be recommended or prescribed a treatment that includes one or more compositions that inhibit ALK kinase activity.

As used herein, the term “subject” refers to a human or non-human mammal or animal. Non-human mammals include livestock animals, companion animals, laboratory animals, and non-human primates. Non-human subjects also specifically include, without limitation, chickens, horses, cows, pigs, goats, dogs, cats, guinea pigs, hamsters, mink, and rabbits. In some embodiments of the invention, a subject is a patient. As used herein, a “patient” refers to a subject who is under the care of a physician or other health care worker, including someone who has consulted with, received advice from or received a prescription or other recommendation from a physician or other health care worker. A patient is typically a subject having or at risk of having NSCLC.

The term “treatment” or “treating” is intended to include prophylaxis, amelioration, prevention or cure of a condition (e.g., NSCLC). Treatment after a condition (e.g., NSCLC) that has started aims to reduce, ameliorate or altogether eliminate the condition, and/or its associated symptoms, or prevent it from becoming worse. Treatment of subjects before a condition (e.g., NSCLC) has started (i.e., prophylactic treatment) aims to reduce the risk of developing the condition and/or lessen its severity if the condition does develop. As used herein, the term “prevent” refers to the prophylactic treatment of a subject who is at risk of developing a condition (e.g., NSCLC) resulting in a decrease in the probability that the subject will develop the disorder, and to the inhibition of further development of an already established disorder.

Aspects of the invention involve inhibiting ALK activity in a cell which expresses an EML4-ALK inversion by contacting the cell with one or more compositions that inhibit ALK kinase activity. Compositions that inhibit ALK kinase activity can be used to treat a disease (e.g., NSCLC) characterized by expression of an EML4-ALK inversion in a subject that has been diagnosed as having an EML4-ALK inversion. It should be appreciated that a composition that inhibits ALK kinase activity may inhibit expression (e.g., transcription, translation, and/or stability) of ALK and/or ALK kinase activity. An inhibitor may be a specific ALK kinase inhibitor or a non-specific inhibitor (e.g., a non-specific kinase inhibitor) or a multi-target inhibitor that inhibits ALK. Inhibitors of ALK kinase have been developed and have been examined in preclinical models. Initial studies have been performed using ALK inhibitors such as WHI-P154 (IC50 ˜5 μM), pyridones (IC50 for staurosporine 0.15-0.78 μM) or with HSP90 inhibitors (Li et al., Med Res Rev, 2007, August 10, Epub ahead of print). Subsequently, more potent and specific ALK inhibitors such as diamino or aminopyrimidines have been developed. These include diamino and aminopyrimidine inhibitors of ALK kinase activity such as NVP-TAE684 and PF-02341066 (Galkin et al., Proc Natl Acad Sci USA, 2007, 104:270-275; Zou et al., Cancer Res, 2007, 67:4408-4417). Both of these inhibitors have good bioavailability and they inhibit ALK kinase activity and growth of NPM-ALK positive lymphoma cells in the low nanomolar range (IC50 for in Karpas 299 cells 2-5 nM and 26 nM for NVP-TAE684 and PF02341066, respectively). PF-02341066 is an inhibitor of both MET and ALK presently in phase I clinical development. Methods described herein encompass the use of any kinase inhibitor that inhibits ALK kinase activity, for treatment of NSCLC in subjects that exhibit an EML4-ALK inversion. In some embodiments the kinase inhibitor is NVP-TAE684 (Galkin et al., Proc Natl Acad. Sci., 2007, 104(1):270-5). In other embodiments the kinase inhibitor is PF-02341066 (Christensen et al., Mol Cancer Ther, 2007, 6(12 Pt 1):3314-22). Downstream targets for ALK kinase activity include phosphatidylinositol 3-kinase (PI3K), STAT3, phospholipase γ, and extracellular regulated kinase 1/2 (ERK1/2) (Amin et al., Blood, 2007, 110:2259-2267). In some embodiments, a subject (e.g., an NSCLC patient) who has a disease (e.g., NSCLC) that exhibits an EML4-ALK fusion in at least some, if not all, of the cancer cells, may be recommended or prescribed a treatment that includes one or more compounds that inhibit a component of a downstream signaling pathway.

In some embodiments a composition that inhibits the activity of ALK kinase may be a small molecule, a peptide, an aptamer, or an antibody. In other embodiments, a composition that inhibits the activity of ALK kinase may be an agent that knocks down expression of ALK. As used herein an agent that knocks down expression of ALK can be any molecule or compound that can inhibit expression of ALK. For example, it could be an RNAi, an antisense RNA, a ribozyme, or any other suitable molecule, or any combination thereof. Studies using shRNA knockdown of ALK in NPM-ALK containing models have shown growth inhibition and apoptosis and suggested that ALK inhibition may be a potentially effective therapeutic strategy (Piva et al., Blood, 2006, 107:689-697).

Various strategies for gene knockdown known in the art can be used to inhibit gene expression (e.g., expression of ALK). For example, gene knockdown strategies may be used that make use of RNA interference (RNAi) and/or microRNA (miRNA) pathways including small interfering RNA (siRNA), short hairpin RNA (shRNA), double-stranded RNA (dsRNA), miRNAs, and other small interfering nucleic acid-based molecules known in the art. Methods for knocking down ALK expression, and examples of suitable molecules for knocking down ALK expression are incorporated by reference from US Patent Publications 20080279870, 20080090776 and 20050005314. Furthermore, one of ordinary skill in the art would be able to design constructs for knocking down expression of ALK without undue experimentation based on the sequence of ALK (GenBank identifier NM_{—}004304). In one embodiment, vector-based RNAi modalities (e.g., shRNA or shRNA-mir expression constructs) are used to reduce expression of a gene (e.g., ALK) in a cell. In some embodiments, therapeutic compositions of the invention comprise an isolated plasmid vector (e.g., any isolated plasmid vector known in the art or disclosed herein) that expresses a small interfering nucleic acid such as an shRNA. The isolated plasmid may comprise a tumor-specific promoter operably linked to a gene encoding the small interfering nucleic acid, e.g., an shRNA. In some cases, the isolated plasmid vector is packaged in a virus capable of infecting the individual. Exemplary viruses include adenovirus, retrovirus, lentivirus, adeno-associated virus, and others that are known in the art and disclosed herein.

A broad range of RNAi-based modalities could be employed to inhibit expression of a gene in a cell, such as siRNA-based oligonucleotides and/or altered siRNA-based oligonucleotides. Altered siRNA based oligonucleotides are those modified to alter potency, target affinity, safety profile and/or stability, for example, to render them resistant or partially resistant to intracellular degradation. Modifications, such as phosphorothioates, for example, can be made to oligonucleotides to increase resistance to nuclease degradation, binding affinity and/or uptake. In addition, hydrophobization and bioconjugation enhances siRNA delivery and targeting (De Paula et al., RNA. 13(4):431-56, 2007) and siRNAs with ribo-difluorotoluoyl nucleotides maintain gene silencing activity (Xia et al., ASC Chem. Biol. 1(3):176-83, (2006)). siRNAs with amide-linked oligoribonucleosides have been generated that are more resistant to S1 nuclease degradation than unmodified siRNAs (Iwase R et al. 2006 Nucleic Acids Symp Ser 50: 175-176). In addition, modification of siRNAs at the 2′-sugar position and phosphodiester linkage confers improved serum stability without loss of efficacy (Choung et al., Biochem. Biophys. Res. Commun. 342(3):919-26, 2006). Other molecules that can be used to inhibit expression of a gene (e.g., ALK gene) include sense and antisense nucleic acids (single or double stranded), ribozymes, peptides, DNAzymes, peptide nucleic acids (PNAs), triple helix forming oligonucleotides, antibodies, and aptamers and modified form(s) thereof directed to sequences in gene(s), RNA transcripts, or proteins. Antisense and ribozyme suppression strategies have led to the reversal of a tumor phenotype by reducing expression of a gene product or by cleaving a mutant transcript at the site of the mutation (Carter and Lemoine Br. J. Cancer. 67(5):869-76, 1993; Lange et al., Leukemia. 6(11):1786-94, 1993; Valera et al., J. Biol. Chem. 269(46):28543-6, 1994; Dosaka-Akita et al., Am. J. Clin. Pathol. 102(5):660-4, 1994; Feng et al., Cancer Res. 55(10):2024-8, 1995; Quattrone et al., Cancer Res. 55(1):90-5, 1995; Lewin et al., Nat. Med. 4(8):967-71, 1998). Ribozymes have also been proposed as a means of both inhibiting gene expression of a mutant gene and of correcting the mutant by targeted trans-splicing (Sullenger and Cech Nature 371(6498):619-22, 1994; Jones et al., Nat. Med. 2(6):643-8, 1996). Ribozyme activity may be augmented by the use of, for example, non-specific nucleic acid binding proteins or facilitator oligonucleotides (Herschlag et al., Embo J. 13(12):2913-24, 1994; Jankowsky and Schwenzer Nucleic Acids Res. 24(3):423-9, 1996). Multitarget ribozymes (connected or shotgun) have been suggested as a means of improving efficiency of ribozymes for gene suppression (Ohkawa et al., Nucleic Acids Symp Ser. (29):121-2, 1993).

Triple helix approaches have also been investigated for sequence-specific gene suppression. Triple helix forming oligonucleotides have been found in some cases to bind in a sequence-specific manner (Postel et al., Proc. Natl. Acad. Sci. U.S.A. 88(18):8227-31, 1991; Duval-Valentin et al., Proc. Natl. Acad. Sci. U.S.A. 89(2):504-8, 1992; Hardenbol and Van Dyke Proc. Natl. Acad. Sci. U.S.A. 93(7):2811-6, 1996; Porumb et al., Cancer Res. 56(3):515-22, 1996). Similarly, peptide nucleic acids have been shown to inhibit gene expression (Hanvey et al., Antisense Res. Dev. 1(4):307-17, 1991; Knudsen and Nielson Nucleic Acids Res. 24(3):494-500, 1996; Taylor et al., Arch. Surg. 132(11):1177-83, 1997). Minor-groove binding polyamides can bind in a sequence-specific manner to DNA targets and hence may represent useful small molecules for suppression at the DNA level (Trauger et al., Chem. Biol. 3(5):369-77, 1996). In addition, suppression has been obtained by interference at the protein level using dominant negative mutant peptides and antibodies (Herskowitz Nature 329(6136):219-22, 1987; Rimsky et al., Nature 341(6241):453-6, 1989; Wright et al., Proc. Natl. Acad. Sci. U.S.A. 86(9):3199-203, 1989). In some cases suppression strategies have led to a reduction in RNA levels without a concomitant reduction in proteins, whereas in others, reductions in RNA have been mirrored by reductions in protein. The diverse array of suppression strategies that can be employed includes the use of DNA and/or RNA aptamers that can be selected to target a protein of interest (e.g., ALK polypeptide).

It should be appreciated that aspects of the invention relate to treating cancers that are characterized by an EML4-ALK fusion, regardless of the expression status of other genes such as PTEN, EGFR or Ras. In some embodiments, an inhibitor of EGFR (e.g., an inhibitor of EGFR activity, expression, etc., or any combination thereof) is also recommended, prescribed, or administered to the subject. Some non-limiting examples of EGFR inhibitors include Erlotinib, Gefitinib, and AG1478. In some embodiments, a chemotherapeutic agent is also recommended, prescribed, and/or administered to the subject. A chemotherapeutic agent may be an alkylating agent, a nucleic acid (e.g., DNA) damaging agent, or other suitable chemotherapeutic agent. In some embodiments, a chemotherapeutic agent is a platinum based compound (e.g., cisplatin or related compound). In some embodiments, a combination of one or more EGFR inhibitors, one or more ALK kinase inhibitors and/or one or more chemotherapeutic agents may be recommended, prescribed, and/or administered to a subject that has been identified as having a condition (e.g., NSCLC) associated with an EML4-ALK fusion.

In some embodiments a subject that has been diagnosed as having an EML4-ALK inversion, is also diagnosed as having an EGFR mutation. In certain embodiments an EGFR mutation is an activating mutation. As used herein an activating mutation in EGFR is any mutation in EGFR that leads to an increase in its activity relative to wildtype EGFR. For example, an activating mutation in EGFR may lead to constitutive activity of EGFR. In some embodiments, a cancer associated with increased EGFR signaling may express a mutated form of EGFR in which there is a deletion within the extracellular domain. In certain embodiments, a mutated form of EGFR is EGFRvIII. In some embodiments, a mutation causing increased activation of EGFR signaling may be caused by a point mutation, deletion, insertion, duplication, inversion or any other mutation, or any combination thereof, in the extracellular domain of EGFR (e.g., in the portion of the EGFR gene encoding the extracellular domain) that gives rise to increased EGFR signaling. A mutation may also be within the intracellular domain of EGFR (e.g., a deletion, point mutation, insertion, duplication, inversion, etc., or any combination thereof) that leads to increased EGFR signaling. A subject that has been diagnosed as having both an EML4-ALK inversion and an EGFR mutation may be treated through administration of one or more compositions that inhibit ALK kinase activity and/or EGFR kinase activity. In some embodiments the subject also undergoes surgery and/or radiation therapy.

In some embodiments the disease that is treated is cancer. A cancer cell is a cell that divides and reproduces abnormally due to a loss of normal growth control. Cancer cells almost always arise from at least one genetic mutation. The term “tumor” is usually equated with neoplasm, which literally means “new growth” and is used interchangeably with “cancer.” A “neoplastic disorder” is any disorder associated with cell proliferation, specifically with a neoplasm. A “neoplasm” is an abnormal mass of tissue that persists and proliferates after withdrawal of the carcinogenic factor that initiated its appearance. There are two types of neoplasms, benign and malignant. Nearly all benign tumors are encapsulated and are noninvasive; in contrast, malignant tumors are almost never encapsulated but invade adjacent tissue by infiltrative destructive growth. This infiltrative growth can be followed by tumor cells implanting at sites discontinuous with the original tumor. The method of the invention can be used to treat neoplastic disorders in humans, including but not limited to: sarcoma, carcinoma, fibroma, leukemia, lymphoma, melanoma, myeloma, neuroblastoma, rhabdomyosarcoma, retinoblastoma, and glioma as well as each of the other tumors described herein.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer; bone cancer; brain and CNS cancer; breast cancer; cervical cancer; choriocarcinoma; colon and rectum cancer; connective tissue cancer; cancer of the digestive system; endometrial cancer; esophageal cancer; eye cancer; cancer of the head and neck; gastric cancer; intra-epithelial neoplasm; kidney cancer; larynx cancer; leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; renal cancer; cancer of the respiratory system; sarcoma; skin cancer; stomach cancer; testicular cancer; thyroid cancer; uterine cancer; cancer of the urinary system, as well as other carcinomas and sarcomas. In some embodiments non small cell lung carcinoma (NSCL) is the cancer being treated or diagnosed.

In one aspect, a method for treating cancer is provided which involves administering the compositions of the invention to a subject having cancer. A “subject having cancer” is a subject that has been diagnosed with a cancer. In some embodiments, the subject has a cancer type characterized by a solid mass tumor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue.

However, some primary tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

As used herein, a “cancer medicament” refers to a agent which is administered to a subject for the purpose of treating a cancer. As used herein, “treating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Various types of medicaments for the treatment of cancer are described herein. Cancer medicaments embrace such categories as chemotherapeutic agents, immunotherapeutic agents, cancer vaccines, hormone therapy, and biological response modifiers. Cancer medicaments also include agents which are administered to a subject in order to reduce the symptoms of a cancer, rather than to reduce the tumor or cancer burden (i.e., the number of cancer or tumor cells) in a subject. One example of this latter type of cancer medicament is a blood transfusion which is administered to a subject having cancer in order to maintain red blood cell and/or platelet levels within a normal range. As an example, in the absence of such transfusion, cancer patients with below normal levels of platelets are at risk of uncontrolled bleeding.

A cancer medicament does not refer to either surgical procedures or radiotherapy aimed at treating cancer. According to various aspects of the invention, some forms of compositions that inhibit ALK kinase activity and or EGFR kinase activity and a cancer medicament may be administered after a surgical procedure and/or radiation therapy aimed at treating a cancer. Surgery and radiation are still commonly used to treat a variety of cancers. In some cases, surgery is also used in a prophylactic manner to reduce the risk that a cancer will develop. As an example of this latter use of surgery, subjects at risk of developing breast cancer, for example, those with a familial disposition to breast cancer, sometimes undergo surgical breast removal (i.e., a mastectomy), in order to reduce the risk of developing the disease. Additionally, as described in the Examples section some patients being tested for EML4-ALK inversion have undergone or are scheduled to undergo surgical removal of NSCL carcinoma.

Additionally, the methods of the invention are intended to embrace the use of more than one cancer medicament along with compositions that inhibit ALK kinase activity and/or EGFR kinase activity. As an example, where appropriate, compositions that inhibit ALK kinase activity and/or EGFR kinase activity may be administered with a both a chemotherapeutic agent and an immunotherapeutic agent. Alternatively, the cancer medicament may embrace an immunotherapeutic agent and a cancer vaccine, or a chemotherapeutic agent and a cancer vaccine, or a chemotherapeutic agent, an immunotherapeutic agent and a cancer vaccine all administered to one subject for the purpose of treating a subject having a cancer or at risk of developing a cancer.

As used herein, chemotherapeutic agents are chemical and biological agents which target cancer cells directly. Some of these agents function to inhibit a cellular activity which the cancer cell is dependent upon for continued survival. Categories of chemotherapeutic agents include alkylating/alkaloid agents, antimetabolites, hormones or hormone analogs, and miscellaneous antineoplastic drugs.

Examples of chemotherapeutic agents which can be used according to the invention include but are not limited to Aminoglutethimide, Asparaginase, Busulfan, Carboplatin, Chlorombucil, Cytarabine HCl, Dactinomycin, Daunorubicin HCl, Estramustine phosphate sodium, Etoposide (VP16-213), Floxuridine, Fluorouracil (5-FU), Flutamide, Hydroxyurea (hydroxycarbamide), Ifosfamide, Interferon Alfa-2a, Alfa-2b, Leuprolide acetate (LHRH-releasing factor analogue), Lomustine (CCNU), Mechlorethamine HCl (nitrogen mustard), Mercaptopurine, Mesna, Mitotane (o.p′-DDD), Mitoxantrone HCl, Octreotide, Plicamycin, Procarbazine HCl, Streptozocin, Tamoxifen citrate, Thioguanine, Thiotepa, Vinblastine sulfate, Amsacrine (m-AMSA), Azacitidine, Erthropoietin, Hexamethylmelamine (HMM), Interleukin 2, Mitoguazone (methyl-GAG; methyl glyoxal bis-guanylhydrazone; MGBG), Pentostatin (2′deoxycoformycin), Semustine (methyl-CCNU), Teniposide (VM-26) and Vindesine sulfate.

Hormone therapy is another therapeutic approach that may be combined with methods of cancer treatment disclosed herein. Hormone therapy refers to the use of hormones or hormone substitutes and derivatives in the treatment of subjects having or at risk of having cancer. Examples include estrogen therapy e.g., diethylstilbestrol and ethinyl estradiol (e.g., for breast cancer and prostate cancer), anti-estrogen therapy e.g., tamoxifen (e.g., for breast cancer), progestin therapy e.g., medroxyprogesterone and megestrol acetate (e.g., for breast cancer and endometrial cancer), androgen blockade e.g., anti-androgens such as flutamide (e.g., for prostate cancer), adrenocorticosteroids including adrenal steroids (e.g., for lymphocytic leukemias and lymphomas), synthetic glucocorticoid therapy e.g., prednisone, methylprednisone, and dexamethasone (e.g., for breast cancer, and some CNS neoplasias), androgens e.g., fluoxymesterone (e.g., for breast cancer), synthetic testosterone analogs, aromatase inhibitor e.g., aminoglutethimide (e.g., for breast cancer), gonadotropin-releasing hormone agonists e.g., leuprolide (e.g., for prostate cancer), somatostatin analogs e.g., octreotide (e.g., for gastric cancer and pancreatic cancers).

Biological response modifiers are agents that alter a subject's response to cancer rather than by direct cytotoxicity of the cancer cells. Examples include cytokines e.g., type I interferons (α and β), type II interferon (γ), interleukins (e.g., IL-2, IL-1α and IL-1β), and TNFα and TNF-β; and hemopoietic growth factors e.g., erythropoietin, GM-CSF, and G-CSF.

Compositions of the invention may be administered in effective amounts. An effective amount is a dosage of the composition of the invention sufficient to provide a medically desirable result. An effective amount means that amount necessary to delay the onset of, inhibit the progression of or halt altogether the onset or progression of the particular condition (e.g., NSCLC) being treated. An effective amount may be an amount that reduces one or more signs or symptoms of the condition (e.g., NSCLC). When administered to a subject, effective amounts will depend, of course, on the particular condition being treated (e.g., the NSCLC), the severity of the condition, individual subject parameters including age, physical condition, size and weight, concurrent treatment, frequency of treatment, and the mode of administration. These factors are well known to those of ordinary skill in the art and can be addressed with no more than routine experimentation.

Actual dosage levels of active ingredients in the compositions of the invention can be varied to obtain an amount of the composition of the invention that is effective to achieve the desired therapeutic response for a particular subject, compositions, and mode of administration. The selected dosage level depends upon the activity of the particular composition, the route of administration, the severity of the condition being treated, the condition, and prior medical history of the subject being treated. However, it is within the skill of the art to start doses of the composition at levels lower than required to achieve the desired therapeutic effort and to gradually increase the dosage until the desired effect is achieved. In some embodiments, lower dosages would be required for combinations of multiple compositions than for single compositions (e.g. a composition that inhibits ALK kinase combined with a composition that inhibits a different kinase). Similarly, lower dosages may be required for multi-target inhibitors that inhibit more than one kinase, than for single-target inhibitors.

The compositions of the invention, can be administered to a subject by any suitable route. For example, the compositions can be administered orally, including sublingually, rectally, parenterally, intracisternally, intravaginally, intraperitoneally, topically and transdermally (as by powders, ointments, or drops), bucally, or nasally. The term “parenteral” administration as used herein refers to modes of administration other than through the gastrointestinal tract, which include intravenous, intramuscular, intraperitoneal, intrasternal, intramammary, intraocular, retrobulbar, intrapulmonary, intrathecal, subcutaneous and intraarticular injection and infusion. Surgical implantation also is contemplated, including, for example, embedding a composition of the invention in the body such as, for example, in the brain, in the abdominal cavity, under the splenic capsule, or in the cornea.

Dosage forms for topical administration of a composition of this invention include powders, sprays, ointments, and inhalants as described herein. The composition is mixed under sterile conditions with a pharmaceutically acceptable carrier and any needed preservatives, buffers, or propellants that may be required.

Pharmaceutical compositions of the invention for parenteral injection comprise pharmaceutically acceptable sterile aqueous or nonaqueous solutions, dispersions, suspensions, or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These compositions also can contain adjuvants such as preservatives, wetting agents, emulsifying agents, and dispersing agents. Prevention of the action of microorganisms can be ensured by the inclusion of various antibacterial and antifungal agents, for example, paraben, chlorobutanol, phenol sorbic acid, and the like. It also may be desirable to include isotonic agents such as sugars, sodium chloride, and the like. Prolonged absorption of the injectable pharmaceutical form can be brought about by the inclusion of agents that delay absorption, such as aluminum monostearate or gelatin.

In some cases, in order to prolong the effect of the composition, it is desirable to slow the absorption of the composition from subcutaneous or intramuscular injection. This result can be accomplished by the use of a liquid suspension of crystalline or amorphous materials with poor water solubility. The rate of absorption of the composition then depends upon its rate of dissolution, which, in turn, may depend upon crystal size and crystalline form. Alternatively, delayed absorption of a parenterally administered composition from is accomplished by dissolving or suspending the composition in an oil vehicle.

Injectable depot forms are made by forming microencapsule matrices of the composition in biodegradable polymers such a polylactide-polyglycolide. Depending upon the ratio of composition to polymer, and the nature of the particular polymer employed, the rate of composition release can be controlled. Examples of other biodegradable polymers include poly(orthoesters) and poly(anhydrides). Depot injectable formulations also are prepared by entrapping the drug in liposomes or microemulsions that are compatible with body tissue.

The injectable formulations can be sterilized, for example, by filtration through a bacterial- or viral-retaining filter, or by incorporating sterilizing agents in the form of sterile solid compositions, which can be dissolved or dispersed in sterile water or other sterile injectable medium just prior to use.

The invention provides methods for oral administration of a pharmaceutical composition of the invention. Oral solid dosage forms are described generally in Remington's Pharmaceutical Sciences, 18th Ed., 1990 (Mack Publishing Co. Easton Pa. 18042) at Chapter 89. Solid dosage forms for oral administration include capsules, tablets, pills, powders, troches or lozenges, cachets, pellets, and granules. Also, liposomal or proteinoid encapsulation can be used to formulate the present compositions (as, for example, proteinoid microspheres reported in U.S. Pat. No. 4,925,673). As is known in the art, liposomes generally are derived from phospholipids or other lipid substances. Liposomes are formed by mono- or multi-lamellar hydrated liquid crystals that are dispersed in an aqueous medium. Any nontoxic, physiologically acceptable, and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p 33, et seq. Liposomal encapsulation may include liposomes that are derivatized with various polymers (e.g., U.S. Pat. No. 5,013,556). In general, the formulation includes a composition of the invention and inert ingredients which protect against degradation in the stomach and which permit release of the biologically active material in the intestine.

In such solid dosage forms, the composition is mixed with, or chemically modified to include, a least one inert, pharmaceutically acceptable excipient or carrier. The excipient or carrier preferably permits (a) inhibition of proteolysis, and (b) uptake into the blood stream from the stomach or intestine. In one embodiment, the excipient or carrier increases uptake of the composition of the invention, overall stability of the composition, and/or circulation time of the composition in the body. Excipients and carriers include, for example, sodium citrate, or dicalcium phosphate, and/or (a) fillers or extenders such as starches, lactose, sucrose, glucose, cellulose, modified dextrans, mannitol, and silicic acid, as well as inorganic salts such as calcium triphosphate, magnesium carbonate and sodium chloride, and commercially available diluents such as FAST-FLO®, EMDEX®, STA-RX 1500®, EMCOMPRESS® and AVICEL®, (b) binders such as, for example, methylcellulose ethylcellulose, hydroxypropylmethyl cellulose, carboxymethylcellulose, gums (e.g., alginates, acacia), gelatin, polyvinylpyrrolidone, and sucrose, (c) humectants, such as glycerol, (d) disintegrating agents, such as agar-agar, calcium carbonate, potato or tapioca starch, alginic acid, certain silicates, sodium carbonate, starch including the commercial disintegrant based on starch, EXPLOTAB®, sodium starch glycolate, AMBERLITE®, sodium carboxymethylcellulose, ultramylopectin, gelatin, orange peel, carboxymethyl cellulose, natural sponge, bentonite, insoluble cationic exchange resins, and powdered gums such as agar, karaya or tragacanth; (e) solution retarding agents such a paraffin, (f) absorption accelerators, such as quaternary ammonium compounds and fatty acids including oleic acid, linoleic acid, and linolenic acid (g) wetting agents, such as, for example, cetyl alcohol and glycerol monosterate, anionic detergent surfactants including sodium lauryl sulfate, dioctyl sodium sulfosuccinate, and dioctyl sodium sulfonate, cationic detergents, such as benzalkonium chloride or benzethonium chloride, nonionic detergents including lauromacrogol 400, polyoxyl 40 stearate, polyoxyethylene hydrogenated castor oil 10, 50 and 60, glycerol monostearate, polysorbate 40, 60, 65, and 80, sucrose fatty acid ester, methyl cellulose and carboxymethyl cellulose; (h) absorbents, such as kaolin and bentonite clay, (i) lubricants, such as talc, calcium sterate, magnesium stearate, solid polyethylene glycols, sodium lauryl sulfate, polytetrafluoroethylene (PTFE), liquid paraffin, vegetable oils, waxes, CARBOWAX® 4000, CARBOWAX® 6000, magnesium lauryl sulfate, and mixtures thereof; (j) glidants that improve the flow properties of the drug during formulation and aid rearrangement during compression that include starch, talc, pyrogenic silica, and hydrated silicoaluminate. In the case of capsules, tablets, and pills, the dosage form also can comprise buffering agents.

Solid compositions of a similar type also can be employed as fillers in soft and hard-filled gelatin capsules, using such excipients as lactose or milk sugar, as well as high molecular weight polyethylene glycols and the like.

The solid dosage forms of tablets, dragees, capsules, pills, and granules can be prepared with coatings and shells, such as enteric coatings and other coatings well known in the pharmaceutical formulating art. They optionally can contain opacifying agents and also can be of a composition that they release the active ingredients(s) only, or preferentially, in a part of the intestinal tract, optionally, in a delayed manner. Exemplary materials include polymers having pH sensitive solubility, such as the materials available as EUDRAGIT® Examples of embedding compositions that can be used include polymeric substances and waxes.

The composition of the invention also can be in microencapsulated form, if appropriate, with one or more of the above-mentioned excipients.

Liquid dosage forms for oral administration include pharmaceutically acceptable emulsions, solutions, suspensions, syrups, and elixirs. In addition to the composition of the invention, the liquid dosage forms can contain inert diluents commonly used in the art, such as, for example, water or other solvents, solubilizing agents and emulsifiers, such as ethyl alcohol, isopropyl alcohol ethyl carbonate ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydroflirfuryl alcohol, polyethylene glycols, fatty acid esters of sorbitan, and mixtures thereof.

Besides inert diluents, the oral compositions also can include adjuvants, such as wetting agents, emulsifying and suspending agents, sweetening, coloring, flavoring, and perfuming agents. Oral compositions can be formulated and further contain an edible product, such as a beverage.

Suspensions, in addition to the composition of the invention, can contain suspending agents such as, for example ethoxylated isostearyl alcohols, polyoxyethylene sorbitol and sorbitan esters, microcrystalline cellulose, aluminum metahydroxide, bentonite, agar-agar, tragacanth, and mixtures thereof.

Also contemplated herein is pulmonary delivery of the composition of the invention. The composition is delivered to the lungs of a mammal while inhaling, thereby promoting the traversal of the lung epithelial lining to the blood stream. See, Adjei et al., Pharmaceutical Research 7:565-569 (1990); Adjei et al., International Journal of Pharmaceutics 63:135-144 (1990) (leuprolide acetate); Braquet et al., Journal of Cardiovascular Pharmacology 13 (supp1.5): s.143-146 (1989) (endothelin-1); Hubbard et al., Annals of Internal Medicine 3:206-212 (1989) (α1-antitrypsin); Smith et al., J. Clin. Invest. 84:1145-1146 (1989) (α1-proteinase); Oswein et al., “Aerosolization of Proteins,” Proceedings of Symposium on Respiratory Drug Delivery II, Keystone, Colo., March, 1990 (recombinant human growth hormone); Debs et al., The Journal of Immunology 140:3482-3488 (1988) (interferon-γ and tumor necrosis factor α) and Platz et al., U.S. Pat. No. 5,284,656 (granulocyte colony stimulating factor).

Contemplated for use in the practice of this invention are a wide range of mechanical devices designed for pulmonary delivery of therapeutic products, including, but not limited to, nebulizers, metered dose inhalers, and powder inhalers, all of which are familiar to those skilled in the art.

Some specific examples of commercially available devices suitable for the practice of the invention are the ULTRAVENT® nebulizer, manufactured by Mallinckrodt, Inc., St. Louis, Mo.; the ACORN II® nebulizer, manufactured by Marquest Medical Products, Englewood, Colo.; the VENTOL® metered dose inhaler, manufactured by Glaxo Inc., Research Triangle Park, N.C.; and the SPINHALER® powder inhaler, manufactured by Fisons Corp., Bedford, Mass.

All such devices require the use of formulations suitable for the dispensing of a composition of the invention. Typically, each formulation is specific to the type of device employed and can involve the use of an appropriate propellant material, in addition to diluents, adjuvants, and/or carriers useful in therapy.

The composition may be prepared in particulate form, preferably with an average particle size of less than 10 μm, and most preferably 0.5 to 5 μm, for most effective delivery to the distal lung.

Carriers include carbohydrates such as trehalose, mannitol, xylitol, sucrose, lactose, and sorbitol. Other ingredients for use in formulations may include lipids, such as DPPC, DOPE, DSPC and DOPC, natural or synthetic surfactants, polyethylene glycol (even apart from its use in derivatizing the inhibitor itself), dextrans, such as cyclodextran, bile salts, and other related enhancers, cellulose and cellulose derivatives, and amino acids.

In addition, the use of liposomes, microcapsules or microspheres, inclusion complexes, or other types of carriers is contemplated.

Formulations suitable for use with a nebulizer, either jet or ultrasonic, typically comprise a composition of the invention dissolved in water at a concentration of about 0.1 to 25 mg of biologically active protein per mL of solution. The formulation also can include a buffer and a simple sugar (e.g., for protein stabilization and regulation of osmotic pressure). The nebulizer formulation also can contain a surfactant to reduce or prevent surface-induced aggregation of the inhibitor composition caused by atomization of the solution in forming the aerosol.

Formulations for use with a metered-dose inhaler device generally comprise a finely divided powder containing the composition of the invention suspended in a propellant with the aid of a surfactant. The propellant can be any conventional material employed for this purpose, such as a chlorofluorocarbon, a hydrochlorofluorocarbon, a hydrofluorocarbon, or a hydrocarbon, including trichlorofluoromethane, dichlorodifluoromethane, dichlorotetrafluoroethanol, and 1,1,1,2-tetrafluoroethane, or combinations thereof. Suitable surfactants include sorbitan trioleate and soya lecithin. Oleic acid also can be useful as a surfactant.

Formulations for dispensing from a powder inhaler device comprise a finely divided dry powder containing the composition of the invention and also can include a bulking agent, such as lactose, sorbitol, sucrose, mannitol, trehalose, or xylitol, in amounts that facilitate dispersal of the powder from the device, e.g., 50 to 90% by weight of the formulation.

Nasal delivery of the composition of the invention also is contemplated. Nasal delivery allows the passage of the composition to the blood stream directly after administering the therapeutic product to the nose, without the necessity for deposition of the product in the lung. Formulations for nasal delivery include those with dextran or cyclodextran. Delivery via transport across other mucous membranes also is contemplated.

Compositions for rectal or vaginal administration are preferably suppositories that can be prepared by mixing the composition of the invention with suitable nonirritating excipients or carriers, such as cocoa butter, polyethylene glycol, or suppository wax, which are solid at room temperature, but liquid at body temperature, and therefore melt in the rectum or vaginal cavity and release the active compound.

Also within the scope of the invention are kits for performing FISH assays on chromosomal preparations to detect an EML4-ALK fusion. An example of such a kit may include a set of nucleic acid probes: a first nucleic acid probe wherein the probe has a label and hybridizes to a chromosome comprising EML4, such that if EML4 has not undergone an inversion the probe will hybridize to the uninverted form of the chromosome comprising EML4, and if EML4 has undergone an inversion the probe will hybridize to the derivative chromosome formed via the inversion; and a second a second nucleic acid probe wherein the probe has a label and hybridizes to a chromosome comprising ALK, such that if ALK has not undergone an inversion the probe will hybridize to the uninverted form of the chromosome comprising ALK, and if ALK has undergone an inversion the probe will hybridize to the derivative chromosome formed via the inversion. The kit may further comprise instructions for use of the first and second probes for performing a fluorescent in situ hybridization (FISH) assay to identify an EML4-ALK inversion within a chromosomal preparation. The kit may further comprise instructions for diagnostic purposes, indicating that a positive identification of an EML4-ALK fusion in a chromosome preparation from a cancer patient indicates a positive diagnosis of NSCLC. The kit may further comprise instructions that indicate that a positive identification of an EML4-ALK fusion in a chromosome preparation from a cancer patient indicates that a patient should be treated with a composition that inhibits ALK kinase activity.

In some embodiments a kit may further comprise a DNA counterstain such as DAPI. In some embodiments a kit may further comprise reagents and buffers including but not limited to hybridization buffers and/or wash buffers. In some embodiments a kit may further comprise mounting media and/or one or more control slides.

In other embodiments a kit of the invention may be useful for determining a treatment regimen for cancer or a precancerous condition. For example a biological sample may be taken from a subject who has NSCLC and tested in vitro for response to a composition that inhibits ALK kinase activity. A positive response to an in vitro assay may be taken as a positive indicator that such a subject would respond to in vivo administration of a composition that inhibits ALK kinase activity. An example of such a kit may include one or more compositions that inhibit ALK kinase activity and instructions for testing the compositions on a biological sample for determining whether the biological sample responds to treatment with compositions that inhibit ALK kinase activity.

Kits of the invention may also be useful for treating cancer. An example of such a kit may include one or more compositions that inhibit ALK kinase activity, and instructions for use of the one or more compositions for treating the cancer. Aspects of the invention relate to co-treatments with one or more of the inhibitors described herein. Accordingly, aspects of the invention relate to kits or compositions comprising combinations of two or more (e.g., 2, 3, 4, 5, 6, 7, 8, 9, 10, or more) inhibitors described herein.

A kit of the invention can include a description of use of the composition for participation in any biological or chemical mechanism disclosed herein. Kits can further include a description of activity of the condition in treating the pathology, as opposed to the symptoms of the condition. That is, a kit can include a description of use of the compositions as discussed herein. A kit also can include instructions for use of a combination of two or more compositions of the invention, or instruction for use of a combination of a composition of the invention and one or more other compounds indicated for determining a treatment regimen for cancer or for treatment of a cancer. Instructions also may be provided for administering the composition by any suitable technique as previously described.

The kits described herein may also contain one or more containers, which may contain a composition and other ingredients as previously described. The kits also may contain instructions for mixing, diluting, and/or administering or applying the compositions of the invention in some cases. The kits also can include other containers with one or more solvents, surfactants, preservative and/or diluents (e.g., normal saline (0.9% NaCl), or 5% dextrose) as well as containers for mixing, diluting or administering the components in a sample or to a subject in need of such treatment.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the composition and the mode of use or administration. Suitable solvents for drug compositions are well known, for example as previously described, and are available in the literature. The solvent will depend on the composition and the mode of use or administration.

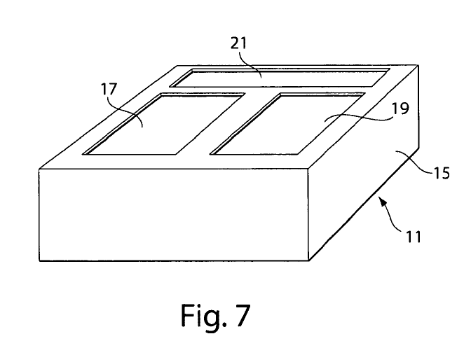

An example of a kit useful according to the invention is shown in FIG. 7. The kit (11) shows a container (15) that houses components such as a set of nucleic acid probes (17, and 19) and instructions for their use (21).

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

### Example 1

### EML4-ALK Inversion and Sensitivity to ALK Kinase Inhibition in Lung Cancer

The EML4-ALK fusion gene, resulting from an inversion on chromosome 2p, has recently been detected in ˜7% of Japanese non-small cell lung cancers (NSCLC). This genetic alteration was also transforming in vitro and in vivo. In the current study the frequency of EML4-ALK in NSCLCs (n=305) derived from US (n=138) and Korean (n=167) patients and in NSCLC cell lines (n=83) was examined. Four different variants of EML4-ALK were detected using RT-PCR in 8 (3%) NSCLC (2/138 (1.5%) from US NSCLC patients; 6/167 (3.6%) from Korean patients) and in 3/83 (3.6%) NSCLC cell lines. All EML4-ALK containing tumors and cell lines were adenocarcinomas and occurred more frequently in NSCLC patients who were never or light (<10 pack years) cigarette smokers compared to current/former smokers (6% vs. 1%; p=0.049). The efficacy of NVP-TAE684, a highly specific ALK kinase inhibitor was examined in NSCLC cell lines with the EML4-ALK inversion. Despite inhibiting ALK phosphorylation in all 3 cell lines, the growth of only 1 of the 3 cell lines, H3122, was inhibited by NVP-TAE684 and this was accompanied by significant apoptosis. In the other 2 EML4-ALK containing cell lines, unlike in H3122, inhibition of ALK phosphorylation led to no or only partial inhibition of AKT, STAT3 or ERK 1/2 phosphorylation. These studies suggest that ALK inhibitors may be potentially effective therapies for a subset of lung cancers harboring the EML4-ALK inversion and further support their clinical development.

### Material and Methods

### Cell Lines and Tumors

Lung tumor cell lines (80 NSCLCs, two mesotheliomas, and one neuroendocrine tumor lines) were purchased from ATCC (Manassas, Va.), or were received from Drs. John D. Minna and Adi F. Gazdar (UT Southwestern, Dallas, Tex.) (Table 1). Two additional NSCLC cell lines (DFCI024, DFCI032) were established at Dana-Farber Cancer Institute from pleural effusions of treatment naïve female NSCLC patients who were never smokers.

NSCLC tumor specimens (n=305) were collected from surgical resections where enough material for RNA extraction was available. The staging of the cancers was done according to the sixth edition of the IASLC staging guidelines. The tumors were categorized by the 2004 WHO classification system. Frozen tumor tissues with a tumor cell content of more than 70% were used for further analysis. In addition, tumor tissues of the following patients were excluded from the study: patients who had received preoperative neoadjuvant treatments, patients who had died within one month from the operation, patients with double primary lung cancer, patients with pleural effusion or pleural seeding, and patients who had undergone incomplete resections or who had not been subjected to mediastinal lymph node dissections. The majority of the specimens (n=167) were collected at the Samsung Medical Center, Korea (Korean cohort) between years 1995-2007, while the rest of the specimens (n=138) were collected at the Brigham and Women's Hospital, Boston, Mass. (U.S. cohort) and have been previously published (14-16).

Tumor and cell line specimens were snap frozen, and stored at −80° C. RNA was extracted using Trizol (Invitrogen, Carlsbad, Calif.) and purified with Rneasy Mini Kit (Qiagen, Valencia, Calif.). 0.5-1 μg of total RNA was used for cDNA synthesis with QuantiTect reverse transcriptation kit (Qiagen, Valencia, Calif.).

TABLE 1
Lung cancer cell lines used in exon array analysis.

| Cell Line | Histology | Gender | Age | Smoker (Y/N/PY) |
|---|---|---|---|---|
| A-427 | Adeno | M | 52 | N/A |
| A549 | Adeno | M | 58 | N/A |
| Calu-1 | NSCLC | M | 47 | N/A |
| Calu-3 | Adeno | M | 25 | N/A |
| Calu-6 | NSCLC | F | 61 | N/A |
| H1299 | LC | M | 43 | N/A |
| H1355 | Adeno | M | 53 | N/A |
| H1395 | Adeno | F | 55 | Y, 15 pack years |
| H1437 | Adeno | M | 60 | Y, 70 pack years |
| H1563 | Adeno | M | N/A | N |
| H1568 | Adeno | F | 48 | Y, 60 pack years |
| H157 | Squamous | N/A | N/A | N/A |
| H1648 | Adeno | M | 39 | Y, 40 pack years |
| H1650 | BA | M | 27 | Y, 10 pack years |
| H1666 | BA | F | 50 | N |
| H1734 | Adeno | F | 56 | N |
| H1755 | Adeno | F | 65 | Y, 60 pack years |
| H1770 | NE | M | 57 | N |
| H1781 | BA | F | 66 | Y, 60 pack years |
| H1792 | Adeno | M | 50 | Y, 30 pack years |
| H1819 | Adeno | F | 55 | Y, 80 pack years |
| H1838 | AD | F | N/A | N |
| H1915 | LC | F | 61 | N |
| H1944 | Adeno | F | 62 | Y, 40 pack years |
| H1975 | Adeno | F | N/A | N |
| H1993 | Adeno | F | 47 | Y, 30 pack years |
| H2009 | Adeno | F | 68 | Y, 30 pack years |
| H2030 | Adeno | M | N/A | N |
| H2052 | ME | M | 65 | Y, 40 pack years |
| H2073 | Adeno | F | 47 | Y, 30 pack years |
| H2087 | Adeno | M | 69 | Y, 60 pack years |
| H2110 | NSCLC | N/A | N/A | N |
| H2122 | Adeno | N/A | N/A | N/A |
| H2126 | LC | M | 65 | N/A |
| H2172 | NSCLC | F | N/A | N |
| H2228 | Adeno | F | N/A | N |
| H23 | Adeno | M | 51 | N/A |
| H2347 | Adeno | F | 54 | N |
| H2444 | NSCLC | M | N/A | N/A |
| H28 | ME | M | 48 | Y, 29 pack years |
| H2882 | NSCLC | N/A | N/A | |
| H2887 | NSCLC | N/A | N/A | Y, 50 pack years |
| H3122 | NSCLC | N/A | N/A | N/A |
| H322 | BA | N/A | N/A | N/A |
| H3255 | Adeno | N/A | N/A | N/A |
| H358 | BA | M | N/A | N/A |
| H441 | Adeno | M | N/A | N/A |
| H460 | LC | M | N/A | N/A |
| H520 | Squamous | M | N/A | N/A |
| H522 | Adeno | M | 60 | N/A |
| H596 | ADSQ | M | 73 | N/A |
| H647 | ADSQ | M | 56 | N |
| H661 | LC | M | 43 | N/A |
| H820 | BA | M | 53 | N/A |
| HCC1171 | NSCLC | N/A | N/A | N/A |
| HCC1195 | Adeno (mixed) | N/A | N/A | N/A |
| HCC1359 | LC | N/A | N/A | N/A |
| HCC15 | Squamous | N/A | N/A | N/A |
| HCC1833 | Adeno | N/A | N/A | N/A |
| HCC193 | Adeno | N/A | N/A | N/A |
| HCC2279 | Adeno | N/A | N/A | N/A |
| HCC2429 | NSCLC | N/A | N/A | N/A |
| HCC2450 | Squamous | N/A | N/A | N/A |
| HCC2935 | NSCLC | N/A | N/A | N/A |
| HCC364 | Adeno | N/A | N/A | N/A |
| HCC366 | ADSQ | N/A | N/A | N/A |
| HCC4006 | Adeno | N/A | N/A | N/A |
| HCC44 | NSCLC | N/A | N/A | N/A |
| HCC461 | Adeno | N/A | N/A | N/A |
| HCC515 | Adeno | N/A | N/A | N/A |
| HCC78 | Adeno | N/A | N/A | N/A |
| HCC827 | Adeno | N/A | N/A | N/A |
| | (BA features) | | | |
| HCC95 | Squamous | N/A | N/A | N/A |
| PC9 | Adeno | N/A | N/A | N/A |
| SK-LU-1 | Adeno | F | 60 | N/A |
| HOP-62 | N/A | N/A | N/A | N/A |
| HOP-92 | N/A | N/A | N/A | N/A |
| LCLC103H | N/A | N/A | N/A | N/A |
| LCLC97TM1 | N/A | N/A | N/A | N/A |
| LouNH91 | N/A | N/A | N/A | N/A |
| Colo699 | N/A | N/A | N/A | N/A |
| DV-90 | N/A | N/A | N/A | N/A |
| EKVX | N/A | N/A | N/A | N/A |

* Adeno = adenocarcinoma; ADSQ = adenosquamous cell carcinoma; BA = brochoalveolar carcinoma; LC = large cell carcinoma; ME = mesothelioma; NE = neuroendocrine carcinoma; NSCLC = non-small cell carcinoma; Squamous = squamous cell carcinoma. N/A: not available

### Exon Array Studies

To screen for ALK translocations in lung cancer cell lines, data of existing Affymetrix HuEx-1.0 Exon Array (Affymetrix, Santa Clara, Calif.) that had been previously generated from mRNA from these cell lines was used. Unlike typical mRNA expression arrays, where probes are restricted to the 3′ end of every transcript, the HuEx-1.0 array was designed to contain probes mapping to every known and predicted exon in the human genome. It was reasoned that translocations in the ALK gene would result in disparate levels of expression between exons 5′ and 3′ of the breakpoint, with the expression higher in the 3′ end (kinase domain). After performing array normalization and background correction for all probes, analysis was restricted to the 104 probes uniquely mapping to the ALK gene (Refseq NM_{—}004304). To correct for differences in probe response characteristics across the gene, for every sample probe intensity value was divided by the average probe intensity across the other wild type specimens. For each cell line, the location of the most likely breakpoint was computed as the probe which gives the maximum deviation between average expression of 5′ and 3′ probe subsets. Significance levels for each inferred breakpoint were computed using a simple two-sided t-test.

### RT-PCR and Mutation Analysis

For RT-PCR analysis of EML4-ALK, the same primer sequences (primer set 1) as used in the paper originally describing EML4-ALK were used (9). In primer set 1, the forward primer is located at exon 13 of EML4 while the reverse primer is located at exon 20 of ALK. EML4-ALK fusion can also occur between exon 6 of EML4 and exon 19 of ALK (variant 3) (Mano H., unpublished), which the primer would be missed by primer set 1 and therefore an additional set of primers (primer set 2) was used which has a forward primer in exon 3 of EML4 (5′-taccagtgctgtctcaattgcagg-3′) (SEQ ID NO:3) and uses the same reverse primer as the primer set 1. PCR amplification was done using JumpStart Taq enzyme (Sigma, St. Louis, Mo.) under manufacturers guidelines. The resulting PCR products were analyzed using agarose gel electrophoresis.

For the NSCLC tumors, genotyping for KRas or EGFR was done using RT-PCR based SURVEYOR-WAVE mutation analysis (17) followed by sequencing of the positive specimens or by direct sequencing of the RT-PCR products. Mutation analysis of DFCI032 cell line was done using a genomic DNA based SURVEYOR-WAVE analysis with primers for specific exons harboring known mutations in EGFR, KRas, B-Raf, PIK3CA, and Her2.

### Fluorescence In Situ Hybridization

Bacterial artificial chromosomes (BAC) RP11-667I6 and RP11-100C1 (Children's Hospital Oakland Research Institute, Oakland, Calif.) were used as probes for the EML4 and ALK genes, respectively. BAC clones were each streaked onto an LB-Agar plate containing chloramphenicol and grown overnight at 37° C. One colony from each BAC clone was selected and grown in TB overnight at 37° C. and BAC DNA was extracted using established methodologies.

Slides for metaphase FISH were prepared using standard cytogenetic methodologies. Paraffin embedded slides were incubated on a 60° C. hot plate overnight and then subjected to two xylene treatments at room temperature for 2 minutes each. Tissues were then placed in 100 mM Tris-base/50 mM EDTA (pH 7.0) at 100° C. for 45 minutes, rinsed in 1×PBS at room temperature for 5-10 minutes and then treated twice with 150 μL of Digest-All (Zymed, San Francisco, Calif.) at 37° C. for 20 minutes. Slides were rinsed in 1×PBS at room temperature for 5-10 minutes and then fixed in 10% formalin at room temperature for 1 minute. Finally, tissues were dehydrated in a 70%, 90%, and 100% ethanol series for 2 minutes each.

RP11-100C1 BAC DNA (1 μg) was labeled with spectrum red dUTP by nick translation (Vysis, Des Plains, Ill.) and 1 μg of RP11-667I6 BAC DNA was labeled with spectrum green-11-dUTP by nick translation (Vysis, Des Plains, Ill.) using the manufacturer's recommended protocols. The probes were combined together along with 50 ug of Cot-1 DNA, ethanol precipitated, and resuspended in 20 ul of hybrizol containing 50% formamide. Probes were hybridized and washed according to standard FISH procedures (18).

### ALK Kinase Inhibitors

NVP-TAE684 is potent and specific ALK kinase inhibitor and was synthesized by Dr. N. Gray at Dana-Farber Cancer Institute (12, 19, 20). NVP-TAE684 was synthesized according to the procedures published in the patent and the structure and purity of the resulting compounds was confirmed using liquid chromatography-electrospray mass spectrometry (LC-MS) and nuclear magnetic resonance (NMR).

### Cell Proliferation and Growth Assays

Growth and inhibition of growth was assessed by methoxy-tetrazolium salt (MTS) assay. This assay, a colorimetric method for determining the number of viable cells, is based on the bioreduction of 3-(4,5-dimethylthiazol-2-yl)-5-(3-carboxymethoxyphenyl)-2-(4-sulfophenyl)-2H-tetrazolium (MTS) by cells to a formazan product that is soluble in cell culture medium, can be detected spectrophotometrically. NSCLC cells were exposed to treatment for 72 hours and the number of cells used per experiment determined empirically. All experimental points were set up in six to twelve wells and all experiments were repeated at least three times. The data was graphically displayed using GraphPad Prism version 3.00 for Windows, (GraphPad Software; available at the GraphPad internet site). The curves were fitted using a non-linear regression model with a sigmoidal dose response.

### Antibodies and Western Blotting

Cells grown under the previously specified conditions were lysed using Cell Lysis Buffer (Cell Signaling Technologies, Danvers, Mass.) under manufacturers guidelines. After cell lysis, lysates were centrifuged at 16,000×g for 10 min at 4° C. The supernatant was used for subsequent procedures. Western blot analyses were conducted after separation by SDS/PAGE electrophoresis and transfer to nitrocellulose membranes. Immunoblotting was performed according to the antibody manufacturers' recommendations. Antibody binding was detected using an enhanced chemiluminescence system (Perkin Elmer, Boston, Mass.). Anti-ALK, anti-phospho-ALK (Tyr-1604), anti-phospho-Akt (Ser-473), anti-Akt, anti-STAT3, anti-phospho STAT3 (Tyr705), anti-PTEN, and anti-PARP antibodies were obtained from Cell Signaling Technology (Danvers, Mass.). Total ERK1/2 and phospho-ERK1/2 (pT185/pY187) antibodies were purchased from Biosource International (Camarillo, Calif.). The anti-α-tubulin antibody was purchased from Sigma-Aldrich (St. Louis, Mo.).

### Fluorescence-Activated Cell Sorting Analysis

Cells were collected and fixed in 40% ethanol for at least 1 hour (or until ready for the experiment) at 4° C. The fixed cells were treated with 0.5 mL of 500 mg/mL RNase A for 45 minutes at 37° C. and stained with 69 mmol/L propidium iodide (in 38 mM sodium citrate) for at least 30 minutes at room temperature in the dark. The stained cells were then analyzed for DNA content in a Becton Dickinson fluorescence-activated cell sorter using both ModFit (Verity Software House, Topsham, Me.) and CellQuest (Becton Dickinson, San Jose, Calif.) programs.

### Results

### Identification of EML4-ALK Fusion Gene in NSCLC Cell Lines

In order to rapidly screen a panel of 83 lung cancer cell lines (Table 1) for potential ALK translocations, existing Affymetrix HuEx-1.0 Exon Array data that had been previously generated from mRNA from these cell lines was used. It was reasoned that translocations in the ALK gene would result in disparate levels of expression between exons 5′ and 3′ of the breakpoint, with the expression higher in the 3′ end (kinase domain). For each cell line, the location of the most likely breakpoint as the probe was computed, which gives the maximum deviation between average expression of 5′ and 3′ probe subsets.

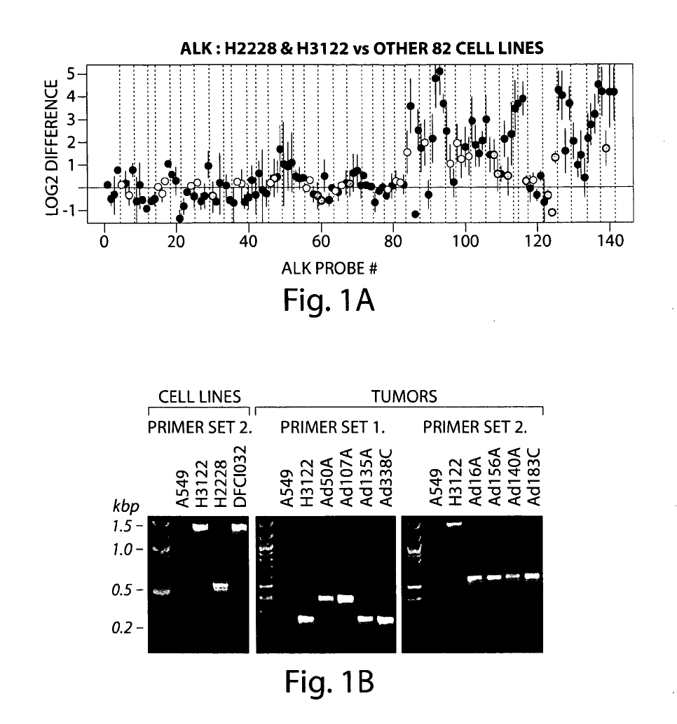

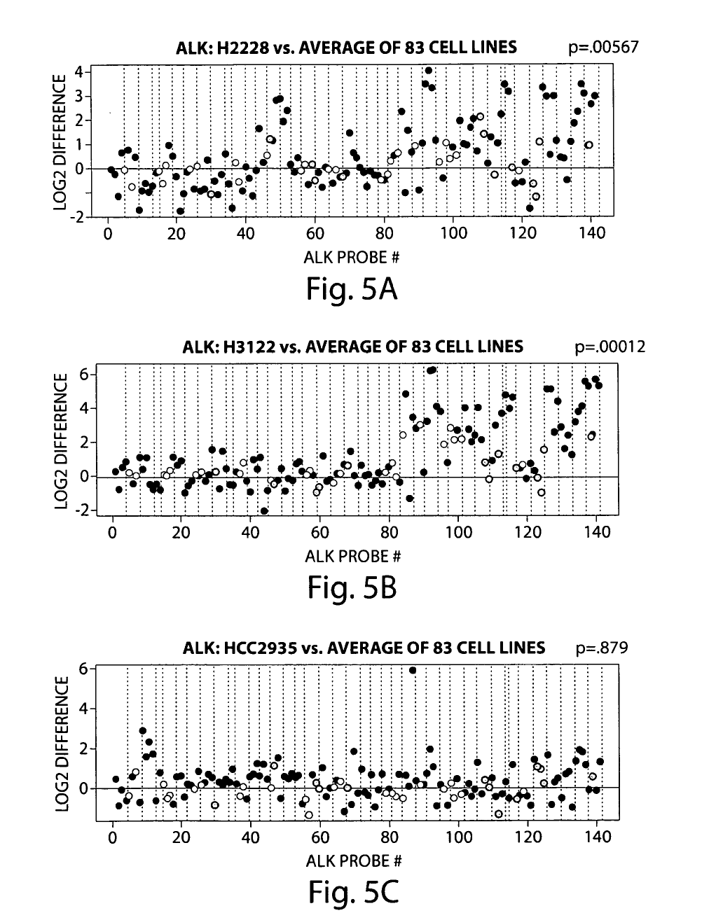

Using this method two cell lines were identified, H3122 and H2228, which had statistically significant (p<0.001) breakpoints in the ALK gene (FIG. 1A and FIG. 5). Although the algorithm used did not consider the location or direction of the breakpoint, the inferred ALK breakpoints in both samples were very near the conserved exon 20 breakpoint in the ALK gene, and in both samples the expression was higher in the 3′ than the 5′ ends. Using RT-PCR, the presence of a fusion gene product was confirmed in both H3122 and H2228 but not in any other of the 81 cell lines. In H3122, RT-PCR primer set 1 (forward primer in exon 13 of EML4, reverse primer in exon 21 of ALK) generated a product of ˜250 bp while in H2228 cell line primer set 2 (forward primer at exon 3 of the EML4, reverse primer in exon 21 of ALK) generated a product of ˜450 bp (FIG. 1B). The RT-PCR results were confirmed by Sanger sequencing, which showed the presence of variant 1 of EML4-ALK in H3122 and variant 3 in H2228 (FIG. 1C). In H2228, the translocation alters the splicing of EML4 part of the gene. An alternatively spliced 33 bp fragment (exon 7a) was detected in this cell line, which was not present in other fusion variants (FIG. 1C, Table 2). Since both H3122 and H2228 cell lines were established from female NSCLC patients with adenocarcinoma histology and the H2228 cell line is from a never-smoker, a screen was conducted for the presence of EML4-ALK in NSCLC cell lines with these clinical features that had been established at Dana-Farber Cancer Institute. 2 cell lines were identified, DFCI024 and DFCI032, both derived from chemotherapy naïve never-smokers with adenocarcinoma. Both cell lines are wild type for EGFR and Kras. With RT-PCR using primer set 2, the EML4-ALK fusion gene was detected in the DFCI032 cell line (FIG. 1B). Thus all together, the EML4-ALK inversion was detected in 3/83 (3.6%) NSCLC cell lines.

TABLE 2
Sequence for alternatively spliced exon 7a of
EML4 detected in fusion variant 3.

(SEQ ID NO: 1)
5′-CAAAAATGTCA ACTCGCGAAAAAAACAGCCAAG-3′

### EML4-ALK in NSCLC Tumors

NSCLC (n=305) tumors from patients of U.S. (n=138) and Korean (n=167) origin were screened using RT-PCR for the EML4-ALK fusion gene. With primer set 1, expression of the fusion gene was detected in four tumors (FIG. 1B). Two of the tumors had RT-PCR products with a size of ˜250 bp while two others had products with a size of ˜450 bp. Tumors with ˜250 bp RT-PCR products were confirmed with Sanger sequencing to have variant 1 of the fusion gene while the tumors with larger products had a previously unpublished variant of the fusion gene (named variant 4 here after). Variant 4 of the fusion gene fuses EML4 codons 1-569 to codons 1078-1621 of ALK (FIG. 1C, Table 3). The tumors were also screened with primer set 2 and an additional 4 tumors were detected to be positive for the genetic alteration (FIG. 1B). All of these were confirmed to contain variant 3 of EML4-ALK by Sanger sequencing. The alternatively spliced exon 7a of EML4 was also present in all the tumors suggesting that this finding was not only limited to H2228 cell line. The tumors also included nine lung metastasis from colon adenocarcinoma and interestingly, one of these contained variant 1 of the EML4-ALK fusion gene.

TABLE 3
The sequence of EML4-ALK variant 4 with
RT-PCR primer set 1.

(SEQ ID NO: 2)
5′TGTGCAGTGTTTAGCATTCTTGGGGAATGGAGATGTTCTTACTGGAGA
CTCAGGTGGAGTCATGCTTATATGGAGCAAAACTACTGTAGAGCCCACAC
CTGGGAAAGGACCTAAAGGTGTATATCAAATCAGCAAACAAATCAAAGCT
CATGATGGCAGTGTGTTCACACTTTGTCAGATGAGAAATGGGATGTTATT
AACTGGAGGAGGGAAAGACAGAAAAATAATTCTGTGGGATCATGATCTGA
ATCCTGAAAGAGAAATAGAGGTTCCTGATCAGTATGGCACAATCAGAGCT
GTAGCAGAAGGAAAGGCAGATCAATTTTTAGTAGGCAAGCTCCGCACCTC
GACCATCATGACCGACTACAACCCCAACTACTGCTTTGCTGGCAAGAA-
3′

The EML4-ALK fusion gene was detected in 8 of 305 (3%) NSCLC tumors (Table 4). Six of the positives (6/167; 3.6%) were detected in tumors from Korean patients while two (2/138 (1.5%)) were detected in NSCLC from U.S. patients. The frequency of EML4-ALK was higher in females (4%) vs. males (2%). NSCLC patients with EML4-ALK containing NSCLC had a younger median age (55.9 y) than patients with wild type tumors (61.9 y). All 8 of the EML4-ALK positive tumors were adenocarcinomas. Furthermore, the fusion gene was detected significantly (p=0.049; Table 4) more frequently in patients (6%; 4/69) with limited smoking history (<10 pack years) compared to tumors from smokers (1%; 2/184). Smoking information was not available from 2 patients with the EML4-ALK inversion. The tumor from 1 of the patients had a concurrent EGFR kinase domain mutation (exon 19 deletion) with the EML4-ALK fusion gene. This patient was treated with surgery alone and has not been treated with either EGFR tyrosine kinase inhibitors gefitinib or erlotinib. None of the 8 tumors contained a concurrent K-Ras mutation.

TABLE 4
Frequency of the EML4-ALK Fusion Gene in NSCLC tumors and its
association with clinical, pathological, and genetic factors.

| Clinical, pathological, and | | EML4-ALK | | | |
|---|---|---|---|---|---|
| genetic characteristics | | + | − | | p-value* |
| All tumors | | 8 (3%) | 297 | (97%) | |
| Ethnicity | U.S. cohort | 2 (1%) | 136 | (99%) | NS |
| | Korean cohort | 6 (3%) | 167 | (97%) | |
| Gender | Male | 3 (2%) | 184 | (98%) | NS |
| | Female | 5 (4%) | 119 | (96%) | |
| Smoking | Never (py <10) | 4 (6%) | 65 | (27%) | 0.049 |
| | Smoker (py >10) | 2 (1%) | 182 | (73%) | |
| Age, median | | 55.9 | 61.9 | | |
| Stage | I | 4 (2%) | 179 | (98%) | |
| | II | 1 (2%) | 58 | (98%) | |
| | III | 3 (6%) | 47 | (94%) | |
| | IV | 0 (0%) | 9 | (100%) | |
| Histology | Adenocarcinoma | 8 (4%) | 200 | (96%) | NS |
| | Squamous carcinoma | 0 (0%) | 88 | (100%) | |
| | Adenosquamous ca | 0 (0%) | 9 | (100%) | |
| Oncogenic | EGFR | 1 (1%) | 68 | (99%) | |
| mutations | K-Ras | 0 (0%) | 49 | (100%) | |

*Fisher's exact test,
NS = not statistically significant (p > 0.05)

### Detection of EML4-ALK Fusion Gene Using FISH

RT-PCR and long range genomic PCR are applicable methods to detect the EML4-ALK fusion gene from cell lines and/or fresh tumor specimens. However, the vast majority of clinical NSCLC tumor samples are formalin fixed paraffin embedded (FFPE) specimens and neither method is feasible to detect the EML4-ALK inversion from FFPEs. In addition, the currently available fluorescence in situ hybridization (FISH) probe for ALK is a break apart probe (Vysis LSI ALK dual color, break apart rearrangement probe, Vysis Inc.) which may not have sufficient resolution to detect a small chromosomal inversion. Thus FISH probes were designed, which hybridize to the 5′ side of the known EML4 breakpoints (green) and the 3′ side of the known ALK breakpoints (red). In the case of wild type genome, the probes should visualize in two separate dots while in the case of the inversion and fusion (any variant), the dots should merge in to single signal (yellow) (FIG. 5).

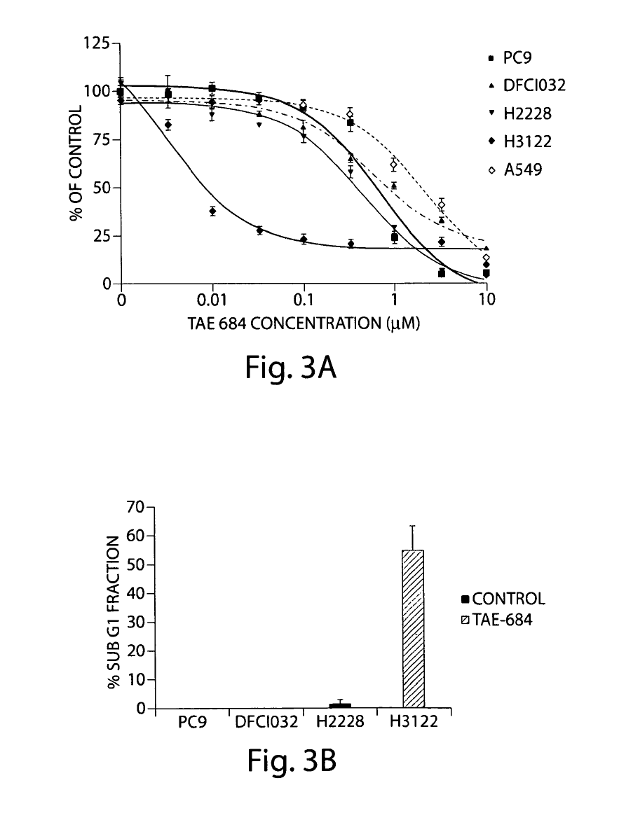

The FISH probes were first examined using NSCLC cell lines with or without EML-ALK fusion gene. In the wild type cell lines PC-9 (FIG. 3A) and A549, the signal for the probes was seen in two separate dots. In DFCI032 (FIG. 3C) and H3122, the probes detected the fusion of the EML4 to ALK in one of the chromosomal pairs while the other pair showed wild type signal. Interestingly, in H2228 cell line, the EML4-ALK fusion gene was detected in small extra-chromosomal fragment (FIG. 3B). Since FISH method was able to detect EML4-ALK fusion in cell lines, it was next investigated whether the method could be used in FFPE specimens. The FFPE tumor specimen, obtained at time of diagnosis from the patient whose pleural effusion was used to establish DFCI032 cell line, was examined. In this FFPE tumor specimen, the EML4-ALK fusion in the interphase nuclei is clearly detectable (FIG. 3D).

### Inhibition of ALK Kinase Activity in EML4-ALK Fusion Gene Containing NSCLC Cell Lines

In the original study describing the EML4-ALK fusion gene, an ALK kinase inhibitor (WHI-154) was shown to induce growth inhibition in the fusion transformed Ba/F3 models (9). Therefore, the effect of ALK kinase inhibitors in the NSCLC cell lines carrying the fusion gene was tested. NVP-TAE684, a highly specific ALK kinase inhibitor (12) was evaluated.

NVP-TAE684 was found to significantly inhibit (IC50 ˜10 nM) the growth of only the H3122 cell line while the other 2 EML4-ALK containing cell lines, H2228 and DFCI032, were as resistant (IC50s 1-10 μM) to the inhibitor as those containing an EGFR mutation (PC-9; delE746_A750) or a K-Ras mutation (A549; G12S) (FIG. 3A). NVP-TAE684 treatment also led to significant apoptosis only in the H3122 cell line as detected by fluorescence activated cell sorting (FACS) (FIG. 3B) or by Western blotting for cleaved PARP (FIG. 3C). No growth arrest or apoptosis was observed in the other cell lines following TAE684 treatment.

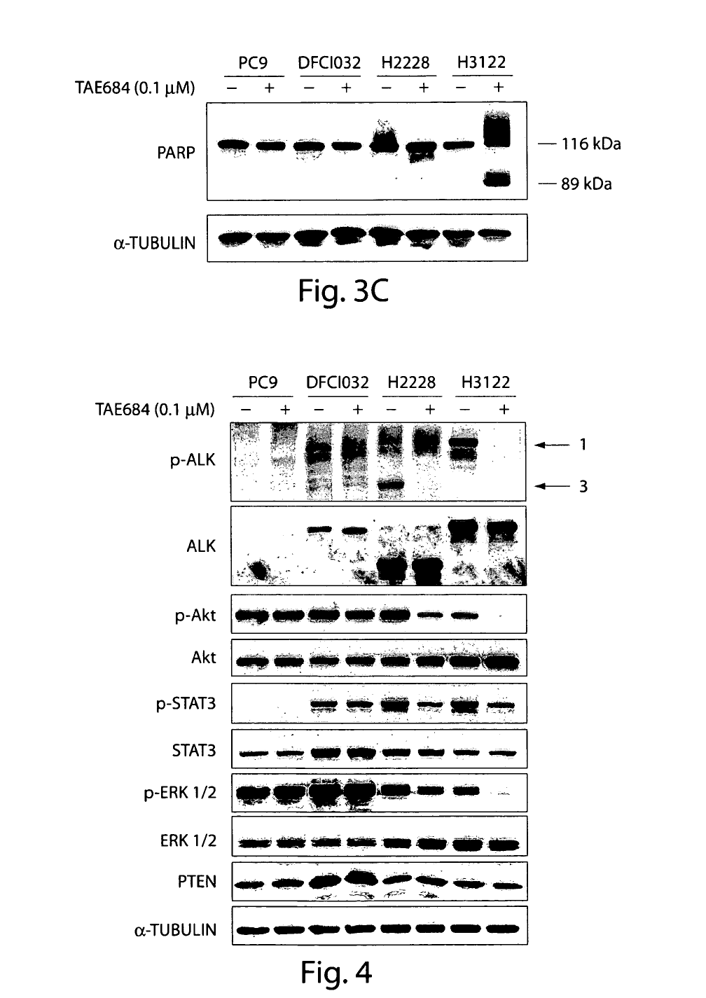

In order to determine why the growth of only 1 of 3 of the EML4-ALK containing cell lines was inhibited by TAE-684, its effects on phosphorylation of ALK and downstream signalling proteins was examined. Both total and phosphorylated ALK were present in all the EML4-ALK positive cell lines (H3122, H2228, and DFCI032) but were absent in the EGFR mutant PC-9 cell line (FIG. 4). Following 0.1 μM NVP-TAE684 treatment, complete downregulation of phoshorylated ALK was observed in all 3 of the EML4-ALK positive cell lines. However, this was accompanied by substantial inhibition of Akt, STAT3 and ERK 1/2 phosphorylation only in the H3122 cell line. In the H2228 cell line there was some but not complete inhibition of Akt and ERK1/2 phosphorylation while these were unchanged in the DFCI032 cell line. A concurrent mutation in EGFR, Kras, B-Raf, HER2 or PIK3CA was not detected in this cell line (data not shown) to account for these findings. Furthermore, the DFCI032 cell line expressed PTEN (FIG. 4). No alterations in the ALK kinase domain sequence in the fusion gene was detected from H2228 or DFCI032 (data not shown).

### Discussion

In the present study the frequency of the EML4-ALK inversion in NSCLC cell lines and primary tumors from NSCLC patients of different ethnic backgrounds was characterized. The EML4-ALK fusion gene was detected in 3% of NSCLC specimens, more frequently in Korean than US NSCLC patients, adenocarcinomas and in patients with limited cigarette smoke exposure. Intriguingly, these same clinicopathological characteristics (female gender, Asian ethnicity, limited cigarette smoking) have been shown to predict for EGFR kinase domain mutations in NSCLC (21). Therefore, the present study provides further evidence for genetic differences in NSCLCs from Caucasian compared with Asian patients and from those who are never or light cigarette smokers. Furthermore, EML4-ALK was detected in 3 NSCLC cell lines including one established (DFCI032) from a previously untreated female never smoker with lung adenocarcinoma. One of the three cell lines with the EML4-ALK translocation (H3122) was also found to be exquisitely sensitive and undergo significant apoptosis following treatment with an ALK kinase inhibitor (NVP-TAE684). The findings in the H3122 cell line suggest the phenomenon of oncogene addiction where ALK kinase solely controls the critical survival signalling pathways in this cell line. ALK inhibition leads to inhibition of all of these signalling pathways and subsequently to apoptosis. This is analogous to EGFR mutant NSCLC (22, 23).

The use of molecular targeted therapy in genetically defined subsets of lung cancer patients is emerging as an effective therapeutic strategy. As an example, 10-30% of NSCLCs contain activating mutations in the EGFR kinase domain and 60-80% of the patients with EGFR mutations obtain dramatic radiographic responses following treatment with the EGFR kinase inhibitors gefitinib or erlotinib (21, 24). Similarly EGFR mutant NSCLC cell lines are exquisitely sensitive to gefitinib in vitro compared with EGFR wild type cell lines and only EGFR mutant NSCLC cell lines undergo apoptosis following gefitinib treatment (22, 25, 26). Findings from this study demonstrate that ALK inhibition may be an effective therapeutic strategy for at least a subset of NSCLC patients whose tumors contain the EML4-ALK fusion gene. Furthermore, as ALK is not normally expressed in the vast majority of adult tissues, specific ALK inhibitors may also be well tolerated. As ALK inhibitors undergo clinical development they should also be examined in NSCLC patients with the EML4-ALK fusion gene. We also developed a FISH assay which can be used to detect the EML4-ALK inversion from FFPE specimens. This will facilitate the identification of appropriate NSCLC patients for clinical studies of ALK kinase inhibitors.

Although 3 NSCLC cell lines with the EML4-ALK inversion were identified, only 1 of the 3 was growth inhibited by TAE684 despite inhibition of ALK phosphorylation in all 3 cell lines (FIGS. 3 and 4). This is quite different from EGFR mutant NSCLC where the majority of EGFR mutant NSCLC cell lines are sensitive to gefitinib or erlotinib in vitro (22, 27-29). These differences may be clinically significant and suggest that ALK inhibitors alone may be effective in a subset of NSCLC patients with the EML4-ALK inversion. In DFCI032, ALK inhibition resulted in no inhibition of downstream signalling proteins (FIG. 4) while in H2228 only partial inhibition of AKT, STAT3 and ERK 1/2 phosphorylation was observed with NVP-TAE684 (FIG. 4). Both DFCI032 or H2228 contain the EML4-ALK translocation in every cell as examined by FISH (data not shown). The mechanism(s) behind this disconnect between inhibition of ALK and inhibition of downstream signalling proteins in the DFCI032 and H2228 cells remains unknown. The DFCI032 and H2228 cell lines do not contain a concurrent mutation in any of the known oncogenes commonly mutated in NSCLC nor have they lost PTEN expression as potential explanations for these observations (30). Alternatively it is possible that these cell lines contain co-activation of other tyrosine kinases leading to parallel survival signalling which may not be inhibited by NVP-TAE684 (31, 32). Recently MET amplification as a mechanism of gefitinib resistance in EGFR mutant NSCLC was described. In the HCC827 GR cell line, which contains both an EGFR mutation and a MET amplification, inhibition of both MET and EGFR was important for growth inhibition and inhibition of Akt phosphorylation (31). Inhibition of EGFR alone or MET alone did not inhibit the growth of HCC827 GR cells nor effect Akt phosphorylation in the samples tested. Further studies of DFCI032 and H2228 may help to determine whether they also contain other concurrently activated kinases. Such studies will hopefully lead to the identification of additional effective combination strategies with NVP-TAE684. Interestingly, a concurrent EGFR activating mutation was detected with the EML4-ALK inversion in 1 of the 8 NSCLC tumor specimens. The clinical significance of these two concurrent mutations on the efficacy of an EGFR kinase inhibitor alone or an ALK kinase inhibitor alone is uncertain. However, as both mutant EGFR and EML4-ALK are oncogenic and activate Akt signalling, a combination strategy of an EGFR inhibitor and an ALK inhibitor would likely be effective to inhibit the growth of this tumor.

### REFERENCES FOR EXAMPLE 1

1. Morris, S. W., Kirstein, M. N., Valentine, M. B., Dittmer, K. G., Shapiro, D. N., Saltman, D. L., and Look, A. T. Science, 263: 1281-1284, 1994.
2. Falini, B., Bigerna, B., Fizzotti, M., Pulford, K., Pileri, S. A., Delsol, G., Carbone, A., Paulli, M., Magrini, U., Menestrina, F., Giardini, R., Pilotti, S., Mezzelani, A., Ugolini, B., Billi, M., Pucciarini, A., Pacini, R., Pelicci, P. G., and Flenghi, L. Am J Pathol, 153:875-886, 1998.
3. Gascoyne, R. D., Lamant, L., Martin-Subero, J. I., Lestou, V. S., Harris, N. L., Muller-Hermelink, H. K., Seymour, J. F., Campbell, L. J., Horsman, D. E., Auvigne, I., Espinos, E., Siebert, R., and Delsol, G. Blood, 102: 2568-2573, 2003.
4. Griffin, C. A., Hawkins, A. L., Dvorak, C., Henkle, C., Ellingham, T., and Perlman, E. J. Cancer Res, 59: 2776-2780, 1999.
5. Lawrence, B., Perez-Atayde, A., Hibbard, M. K., Rubin, B. P., Dal Cin, P., Pinkus, J. L., Pinkus, G. S., Xiao, S., Yi, E. S., Fletcher, C. D., and Fletcher, J. A. Am J Pathol, 157: 377-384, 2000.
6. Amin, H. M. and Lai, R. Blood, 110: 2259-2267, 2007.
7. Iwahara, T., Fujimoto, J., Wen, D., Cupples, R., Bucay, N., Arakawa, T., Mori, S., Ratzkin, B., and Yamamoto, T. Oncogene, 14: 439-449, 1997.
8. Lamant, L., Pulford, K., Bischof, D., Morris, S. W., Mason, D. Y., Delsol, G., and Mariame, B. Am J Pathol, 156: 1711-1721, 2000.
9. Soda, M., Choi, Y. L., Enomoto, M., Takada, S., Yamashita, Y., Ishikawa, S., Fujiwara, S., Watanabe, H., Kurashina, K., Hatanaka, H., Bando, M., Ohno, S., Ishikawa, Y., Aburatani, H., Niki, T., Sohara, Y., Sugiyama, Y., and Mano, H. Nature, 448: 561-566, 2007.
10. Piva, R., Chiarle, R., Manazza, A. D., Taulli, R., Simmons, W., Ambrogio, C., D'Escamard, V., Pellegrino, E., Ponzetto, C., Palestro, G., and Inghirami, G. Blood, 107:689-697, 2006.
11. Li, R. and Morris, S. W. Med Res Rev, 2007.
12. Galkin, A. V., Melnick, J. S., Kim, S., Hood, T. L., Li, N., Li, L., Xia, G., Steensma, R., Chopiuk, G., Jiang, J., Wan, Y., Ding, P., Liu, Y., Sun, F., Schultz, P. G., Gray, N. S., and Warmuth, M. Proc Natl Acad Sci USA, 104: 270-275, 2007.
13. Zou, H. Y., Li, Q., Lee, J. H., Arango, M. E., McDonnell, S. R., Yamazaki, S., Koudriakova, T. B., Alton, G., Cui, J. J., Kung, P. P., Nambu, M. D., Los, G., Bender, S. L., Mroczkowski, B., and Christensen, J. G. Cancer Res, 67: 4408-4417, 2007.
14. Bhattacharjee, A., Richards, W. G., Staunton, J., Li, C., Monti, S., Vasa, P., Ladd, C., Beheshti, J., Bueno, R., Gillette, M., Loda, M., Weber, G., Mark, E. J., Lander, E. S., Wong, W., Johnson, B. E., Golub, T. R., Sugarbaker, D. J., and Meyerson, M. Proc Natl Acad Sci USA, 98: 13790-13795, 2001.
15. Naoki, K., Chen, T. H., Richards, W. G., Sugarbaker, D. J., and Meyerson, M. Cancer Res, 62: 7001-7003, 2002.
16. Hayes, D. N., Monti, S., Parmigiani, G., Gilks, C. B., Naoki, K., Bhattacharjee, A., Socinski, M. A., Perou, C., and Meyerson, M. J Clin Oncol, 24: 5079-5090, 2006.
17. Janne, P. A., Borras, A. M., Kuang, Y., Rogers, A. M., Joshi, V. A., Liyanage, H., Lindeman, N., Lee, J. C., Halmos, B., Maher, E. A., Distel, R. J., Meyerson, M., and Johnson, B. E. Clin Cancer Res, 12: 751-758, 2006.
18. Lee, C., Critcher, R., Zhang, J. G., Mills, W., and Farr, C. J. Chromosoma, 109: 381-389, 2000.
19. Garcia-Echeverria, C., Kanazawa, T., Kawahara, E., Masuya, K., Matsuura, N., Miyake, T., Ohmori, O., Umemura, I., Steensma, R., Chopiuk, G., Jiang, J., Wan, Y., Ding, Q., Zhang, Q., Gray, N. S., and Karanewsky, D. United States patent
20. Garcia-Echeverria, C., Kanazawa, T., Kawahara, E., Masuya, K., Matsuura, N., Miyake, T., Ohmori, O., and Umemura, I. United States patent WO2004080980, 2004.
21. Janne, P. A. and Johnson, B. E. Clin Cancer Res, 12: 4416s-4420s, 2006.
22. Tracy, S., Mukohara, T., Hansen, M., Meyerson, M., Johnson, B. E., and Janne, P. A. Cancer Res, 64: 7241-7244, 2004.
23. Sordella, R., Bell, D. W., Haber, D. A., and Settleman, J. Science, 305: 1163-1167, 2004.
24. Inoue, A., Suzuki, T., Fukuhara, T., Maemondo, M., Kimura, Y., Morikawa, N., Watanabe, H., Saijo, Y., and Nukiwa, T. J Clin Oncol, 24: 3340-3346, 2006.
25. Paez, J. G., Jaime, P. A., Lee, J. C., Tracy, S., Greulich, H., Gabriel, S., Herman, P., Kaye, F. J., Lindeman, N., Boggon, T. J., Naoki, K., Sasaki, H., Fujii, Y., Eck, M. J., Sellers, W. R., Johnson, B. E., and Meyerson, M. Science, 304: 1497-1500, 2004.
26. Mukohara, T., Civiello, G., Johnson, B. E., and Janne, P. A. Oncology, 68: 500-510, 2005.
27. Mukohara, T., Engelman, J. A., Hanna, N. H., Yeap, B. Y., Kobayashi, S., Lindeman, N., Halmos, B., Pearlberg, J., Tsuchihashi, Z., Cantley, L. C., Tenen, D. G., Johnson, B. E., and Janne, P. A. J Natl Cancer Inst, 97: 1185-1194, 2005.
28. Amann, J., Kalyankrishna, S., Massion, P. P., Ohm, J. E., Girard, L., Shigematsu, H., Peyton, M., Juroske, D., Huang, Y., Stuart Salmon, J., Kim, Y. H., Pollack, J. R., Yanagisawa, K., Gazdar, A., Minna, J. D., Kurie, J. M., and Carbone, D. P. Cancer Res, 65: 226-235, 2005.
29. Zhou, B. B., Peyton, M., He, B., Liu, C., Girard, L., Caudler, E., Lo, Y., Baribaud, F., Mikami, I., Reguart, N., Yang, G., Li, Y., Yao, W., Vaddi, K., Gazdar, A. F., Friedman, S. M., Jablons, D. M., Newton, R. C., Fridman, J. S., Minna, J. D., and Scherle, P. A. Cancer Cell, 10: 39-50, 2006.
30. Nagata, Y., Lan, K. H., Zhou, X., Tan, M., Esteva, F. J., Sahin, A. A., Klos, K. S., Li, P., Monia, B. P., Nguyen, N. T., Hortobagyi, G. N., Hung, M. C., and Yu, D. Cancer Cell, 6:117-127, 2004.
31. Engelman, J. A., Zejnullahu, K., Mitsudomi, T., Song, Y., Hyland, C., Park, J. O., Lindeman, N., Gale, C. M., Zhao, X., Christensen, J., Kosaka, T., Holmes, A. J., Rogers, A. M., Cappuzzo, F., Mok, T., Lee, C., Johnson, B. E., Cantley, L. C., and Janne, P. A. Science, 316: 1039-1043, 2007.
32. Stommel, J. M., Kimmelman, A. C., Ying, H., Nabioullin, R., Ponugoti, A. H., Wiedemeyer, R., Stegh, A. H., Bradner, J. E., Ligon, K. L., Brennan, C., Chin, L., and DePinho, R. A. Science, 318: 287-290, 2007.

What is claimed is:

1. A method comprising performing a fluorescent in situ hybridization (FISH) assay to identify an EML4-ALK inversion within a chromosomal preparation comprising:
(a) contacting, under hybridization conditions, a chromosomal preparation with a set of probes comprising a first nucleic acid probe and a second nucleic acid probe; wherein the first probe has a first label and is hybridizable to an uninverted form of the first chromosome; wherein the second probe has a second label different from the first label and is hybridizable to an uninverted form of the second chromosome; wherein (i) if the first and second chromosomes have undergone an inversion and fusion, the first and second probes hybridize to a derivative chromosome formed via the inversion and fusion, such that both the first and second labels appear as a single signal; whereas (ii) if the first and second chromosomes have not undergone the inversion and fusion, the first and second probes hybridize to their respective chromosomes, such that two signals are detected;
(b) detecting a pattern of hybridization for the first and second probes; and
(c) determining from the pattern whether the first and second probes appear on the derivative chromosome or appear separately on the first and second chromosomes, respectively, thereby determining the presence or absence of the EML4-ALK inversion.

2. The method of claim 1, wherein each probe is hybridizable to an uninverted form of each chromosome at a region located within 5 Mb, 2 Mb or 1 Mb of the breakpoint associated with the inversion.

3-14. (canceled)

15. The method of claim 1, wherein the method is a method for determining whether a subject with non-small cell lung cancer should be treated with a composition that inhibits ALK kinase activity.

16. The method of claim 15 further comprising treating the subject with a composition that inhibits ALK kinase activity.

17. The method of claim 15 wherein each probe is hybridizable to an uninverted form of each chromosome at a region located within 5 Mb, 2 Mb or 1 Mb of the breakpoint associated with the inversion.

18-19. (canceled)

20. The method of claim 15 wherein the first nucleic acid probe comprises at least 80% sequence identity with the sequence of RP11-667I6 and has a first label, and the second nucleic acid probe comprises at least 80% sequence identity with the sequence of RP11-100C1, and has a second label.

21. The method of claim 16 wherein the composition that inhibits ALK kinase activity comprises a kinase inhibitor.

22. The method of claim 21 wherein the kinase inhibitor is NVP-TAE684.

23. The method of claim 21 wherein the kinase inhibitor is PF-02341066.

24. The method of claim 16 wherein the composition that inhibits ALK kinase activity comprises an agent that knocks down expression of ALK.

25. The method of claim 24 wherein the composition that inhibits ALK kinase activity comprises an antisense RNA, an RNAi, a ribozyme, or any combination thereof.

26. The method of claim 16 wherein the composition that inhibits ALK kinase activity comprises an antibody, a small molecule, a peptide, an aptamer or any combination thereof.

27. The method of claim 16 further comprising administering an EGFR inhibitor to the subject.

28. A nucleic acid probe for detecting a chromosomal inversion between EML4 and ALK, wherein the probe has a label and hybridizes to a chromosome comprising EML4, such that if EML4 has not undergone an inversion the probe will hybridize to the uninverted form of the chromosome comprising EML4, and if EML4 has undergone an inversion the probe will hybridize to the derivative chromosome formed via the inversion.

29. A composition comprising the nucleic acid probe of claim 28 and further comprising a second probe having a label that hybridizes to a chromosome comprising ALK, such that if ALK has not undergone an inversion the second probe will hybridize to the uninverted form of the chromosome comprising ALK, and if ALK has undergone an inversion the second probe will hybridize to the derivative chromosome formed via the inversion.

30. The probe of claim 28 wherein the probe is hybridizable to an uninverted form of the chromosome at a region located within 5 Mb, 2 Mb or 1 Mb of the breakpoint associated with the inversion.

31-38. (canceled)

39. A kit for identifying an EML4/ALK inversion within a chromosomal preparation, the kit comprising:
(a) a first nucleic acid probe wherein the probe has a label and hybridizes to a chromosome comprising EML4, such that if EML4 has not undergone an inversion the probe will hybridize to the uninverted form of the chromosome comprising EML4, and if EML4 has undergone an inversion the probe will hybridize to the derivative chromosome formed via the inversion;
(b) a second nucleic acid probe wherein the probe has a label and hybridizes to a chromosome comprising ALK, such that if ALK has not undergone an inversion the probe will hybridize to the uninverted form of the chromosome comprising ALK, and if ALK has undergone an inversion the probe will hybridize to the derivative chromosome formed via the inversion;
(c) instructions for use of the first and second probes for performing a fluorescent in situ hybridization (FISH) assay to identify an EML4-ALK inversion within a chromosomal preparation.

40-69. (canceled)

70. The method of claim 1, wherein the method is a method for determining the presence or absence of an EML4-ALK inversion.

71. The method of claim 1 wherein the first nucleic acid probe comprises at least 80% sequence identity with the sequence of RP11-667I6 and has a first label, and the second nucleic acid probe comprises at least 80% sequence identity with the sequence of RP11-100C1, and has a second label.

72. The method of claim 1 wherein the first nucleic acid probe is RP11-667I6, and has a label, and the second nucleic acid probe is RP11-100C1, and has a second label.


SEQUENCE LISTING (3 sequences)

SEQ ID NO: 1 - DNA, 33 bp, Homo sapiens
caaaaatgtcaactcgcgaaaaaaacagccaag

SEQ ID NO: 2 - DNA, 396 bp, Homo sapiens
tgtgcagtgtttagcattcttggggaatggagatgttcttactggagactcaggtggagtcatgcttatatggagcaaaactactgtagagcccacacctgggaaaggacctaaaggtgtatatcaaatcagcaaacaaatcaaagctcatgatggcagtgtgttcacactttgtcagatgagaaatgggatgttattaactggaggagggaaagacagaaaaataattctgtgggatcatgatctgaatcctgaaagagaaatagaggttcctgatcagtatggcacaatcagagctgtagcagaaggaaaggcagatcaatttttagtaggcaagctccgcacctcgaccatcatgaccgactacaaccccaactactgctttgctggcaagaa

SEQ ID NO: 3 - DNA, 24 bp, artificial sequence
  synthetic oligonucleotide
taccagtgctgtctcaattgcagg